(19) United States
(12) Patent Application Publication
(10) Pub. No.: US 2007/0178510 A1
(43) Pub. Date: Aug. 2, 2007

(54) METHODOLOGIES FOR THE DIAGNOSIS AND TREATMENT OF GASTROESOPHAGEAL REFLUX DISEASE

(76) Inventor: Parakrama Chandrasoma, Pasadena, CA (US)

Correspondence Address: CHRISTIE, PARKER & HALE, LLP, PO BOX 7068, PASADENA, CA, 91109-7068, US

(21) Appl. No.: 11/669,059

(22) Filed: Jan. 30, 2007

Related U.S. Application Data
(60) Provisional application No. 60/763,649, filed on Jan. 30, 2006.

(51) Int. Cl. C12Q 1/68; G01N 33/48

(52) U.S. Cl. 435/6; 435/40.5

(57) ABSTRACT

A novel methodology for diagnosing and treating gastroesophageal reflux disease is provided. These methodologies are based on the discovery of a new disease mechanism called hereinafter “reflux carditis”, which is a novel mechanistic view of reflux disease that recognizes cardiac mucosa, which has heretofore been regarded as a normal epithelium in the stomach and an abnormal epithelium in the esophagus, as an abnormal mucosa in both the esophagus and stomach.

### CROSS-REFERENCE TO RELATED APPLICATIONS

The current application claims priority to U.S. Provisional Application No. 60/763,649, filed Jan. 30, 2006, the disclosure of which is incorporated herein by reference.

### FIELD OF THE INVENTION

The present invention relates in general to methods for the diagnosis and treatment of gastroesophageal reflux disease, and more specifically to a novel mechanistic understanding of gastroesophageal reflux disease as well as genetic markers and materials for altering the pathophysiology of gastroesophageal reflux disease.

### BACKGROUND OF THE INVENTION

Few diseases have caused as much confusion and controversy as the columnar lined esophagus that bears the name of Norman Barrett.

Barrett began its eventful history in 1950 when its namesake proclaimed that it did not exist, declaring that the esophagus should be defined as “that part of the foregut, which is lined by squamous epithelium.” (Barrett N R. Chronic peptic ulcer of the oesophagus and “oesophagitis”. Br J Surg 1950:38:175-182, the disclosure of which is incorporated herein by reference.) Allison, who described reflux esophagitis in 1948 and columnar lined esophagus in 1953, in contrast got it almost perfectly right on his first attempt. (See, e.g., Allison P R. Peptic ulcer of the esophagus. Thorax 1948; 3:20-42; and Allison P R, Johnstone A S. The oesophagus lined with gastric mucous membrane. Thorax 1953; 8:87-101, the disclosures of which are incorporated herein by reference.) He accurately interposed a columnar lined distal esophagus between the squamo-columnar junction and the proximal stomach. In 1957, Barrett's reversed his opinion and agreed with Allison that the tubular structure distal to the squamo-columnar junction was a columnar lined esophagus (Barrett N R. The lower esophagus lined by columnar epithelium. Surgery 1957; 41:881-894, the disclosure of which is incorporated herein by reference.) When these classical papers are reviewed, it is incomprehensible that this entity came to be known as Barrett esophagus and not Allison esophagus.

A little recognized fact is that when Norman Barrett reversed himself in 1957, the only histologic definition of the esophagus that has ever existed disappeared. Although the definition was wrong, Barrett's idea was correct: without a histologic definition of the esophagus, gastroesophageal junction and stomach, confusion would surely reign. Indeed, confusion has certainly reined supreme in the 50+ years after Allison. To date, pathologists have no accepted method of accurately defining the demarcation between the esophagus and stomach. Instead, most of us depend on the endoscopist to tell us the location of the gastroesophageal junction and use this information to decide whether a given biopsy sample is from the esophagus or the stomach.

Confusion regarding the diagnosis of Barrett esophagus exists because of a false dogma that cardiac mucosa is normally present in the gastroesophageal junctional region. In fact, this view of what constitutes the normal and healthy functioning of the esophagus is generally well-accepted by the majority of physicians. Accordingly, a new treatment methodology is needed that can provide histolopathologic precision that cannot be matched by any other modality and can convert the confusion that exists regarding diagnosis of Barrett esophagus to complete lucidity in a manner that is simple, accurate, and reproducible.

### SUMMARY OF THE INVENTION

The present invention is directed generally to methods for the diagnosis and treatment of gastroesophageal reflux disease, and more specifically to a novel mechanistic understanding of gastroesophageal reflux disease as well as genetic markers and materials for altering the pathophysiology of gastroesophageal reflux disease. The current invention is based at a fundamental understanding that the only normal epithelia in the esophagus and proximal stomach are squamous epithelium and gastric oxyntic mucosa.

In one embodiment, the invention is directed to precise histologic definitions for the normal state (presence of only squamous and oxyntic mucosa), metaplastic esophageal columnar epithelium (cardiac mucosa with and without intestinal metaplasia, and oxynto-cardiac mucosa), the gastroesophageal junction (the proximal limit of gastric oxyntic mucosa), the esophagus (that part of the foregut lined by squamous and metaplastic columnar epithelium), reflux disease (the presence of metaplastic columnar epithelium), and Barrett esophagus (cardiac mucosa with intestinal metaplasia).

In another embodiment of the invention, a diagnostic methodology is provided for reflux disease. In this embodiment, diagnosis is based on the presence of cardiac mucosa, which is indicative of esophageal epithelium not a normal mucosal type.

In still another embodiment, the invention is directed to a method of determining the severity of reflux which is directly proportional to the amount of metaplastic columnar epithelium, and the risk of adenocarcinoma, which is related to the amount of dysplasia in intestinal metaplastic epithelium present within the columnar lined segment of the esophagus.

In yet another embodiment of the invention, a diagnostic methodology for classifying reflux disease is provided based on the novel categorization of different columnar epithelial types. In such an embodiment, it is provided that cardiac mucosa transforms into oxyntocardiac and intestinal epithelia in the esophagus.

In still yet another embodiment of the invention, a diagnostic methodology for classifying reflux disease is provided based on the recognition that the proximal stomach is properly identified as a dilated end-stage esophagus.

In still yet another embodiment of the invention, a diagnostic methodology for identifying and classifying reflux disease is provided based on genetic changes that accompany different epithelial transformations. In such an embodiment, a genetic change is particularly noted for the transformation of cardiac mucosa to oxyntocardiac and intestinal epithelia in the esophagus.

In still yet another embodiment of the invention, a diagnostic methodology for identifying and classifying reflux disease based on the molecular basis of genetic changes that cause changes in cardiac mucosa to oxyntocardiac and intestinal epithelium.

In still yet another embodiment, the invention is also directed to novel treatment methods based on the control and treatment of the molecular and genetic changes that alter the cardiac mucosa in a patient diagnosed with reflux disease.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1, provides a photographic illustration of a stratified squamous epithelium of the esophagus stained by immunoperoxidase technique for Ki67. This shows a proliferative zone in the suprabasal region where the stem cells are located.

FIG. 2, provides a photographic illustration of a cardiac mucosa at the squamo-columnar junction showing a columnar epithelium composed entirely of mucous cells with no parietal or goblet cells. There is marked elongation and serration of the foveolar region and severe chronic inflammation. This is the typical appearance of severe reflux carditis (H&E).

FIG. 3, provides a photographic illustration of an oxynto-cardiac mucosa showing mild chronic inflammation and short, slightly distorted glands containing a mixture of mucous and parietal cells. There is a small focus of serous/pancreatic cells in the center (H&E).

FIG. 4, provides a photographic illustration of an intestinal metaplasia showing typical goblet cells. At the right of the picture is one foveolar complex that shows no intestinal metaplasia, being composed of mucous cells only indicating that intestinal metaplasia is occurring in cardiac mucosa (H&E).

FIG. 5, provides a photographic illustration of a normal gastric oxyntic mucosa showing a short foveolar pit from which arises a straight tubular gland containing parietal and chief cells without mucous cells. Note the absence of inflammation (H&E).

FIG. 6, provides a flow-chart illustrating the reflux-adenocarcinoma sequence. The first step is the metaplasia of squamous to cardiac mucosa. Depending on the damage environment, further genetic switches are expressed, one leading to the occurrence of parietal cells (oxynto-cardiac mucosa), which is benign and the other leading to the occurrence of goblet cells (intestinal metaplasia). Mutagenic influences act only on the intestinal metaplastic epithelium leading to dysplasia and adenocarcinoma.

FIG. 7, provides a photographic illustration of mild reflux disease. On the right is normal gastric oxyntic mucosa, the proximal limit of which represents the gastroesophageal junction. On the right is the squamo-columnar junction, which has moved cephalad because of columnar metaplasia. Between the two is metaplastic cardiac and oxynto-cardiac mucosa, characterized by inflammation and foveolar hyperplasia (reflux carditis) (H&E).

FIG. 8, provides a photographic illustration of a short segment Barrett esophagus. The tops of the rugal folds are seen at the end of the tubular esophagus. The squamo-columnar junction has moved cephalad in an irregular manner. The intervening metaplastic columnar epithelium is flat, erythematous and shows squamous islands.

FIG. 9, provides a photographic illustration of a long segment Barrett esophagus showing a longer segment of flat metaplastic columnar epithelium separating the proximal limit of the rugal folds and the squamo-columnar junction located as an irregular line in the uppermost region of the esophagus.

FIG. 10, provides a photographic illustration of a normal histologic state characterized by the junction of squamous epithelium (which in this patient lines the entire esophagus) with gastric oxyntic mucosa which lines the stomach. The gastroesophageal and squamo-columnar junctions are coincidental (H&E).

FIG. 11, provides a diagram illustrating the range of expression of differentiation seen during life in a progenitor cell in the distal esophagus. In the normal fetus, this cell normally shows three types of columnar epithelial differentiation before finally suppressing all these differentiating signals and producing stratified squamous epithelium. During life, in patients who have reflux disease, other differentiating signals are activated which result in different types of metaplastic columnar epithelia.

FIG. 12, provides a diagram illustrating the relationship of different epithelial types in the esophagus to adenocarcinoma. As shown, carcinogenesis occurs only in intestinal metaplastic (Barrett) epithelium.

FIG. 13, provides a diagram that illustrates the emergence of a cell surface receptor consequent on a CDX genetic activation that is hypothesized to be the differentiating genetic signal for intestinal metaplasia. As shown, the progenitor cell in the esophagus becomes susceptible to carcinogenesis only when it undergoes metaplasia to intestinal metaplasia.

FIG. 14, provides a diagram of the reflux-adenocarcinoma sequence.

FIG. 15, provides a typical time line for the changes in a patient with reflux. As shown, the onset of reflux is at x years. This time line varies infinitely in different patients but it is likely that most patients have significant intervals between the onset of cardiac metaplasia and intestinal metaplasia and between intestinal metaplasia and adenocarcinoma.

FIG. 16, provides a diagram illustrating the three main events necessary for the development of adenocarcinoma. As shown, the elements that cause damage are all in the gastric juice and reach the esophagus by reflux. For damage (probably acid-induced) and carcinogenesis (dose dependent), there is a gradient that is maximal at the gastroesophageal junction and decreases more proximally. In contrast, the tendency to cause intestinal metaplasia is maximal in the more proximal esophagus and decreases toward the gastroesophageal junction.

FIG. 17, provides schematic diagrams of the esophagus of four different patients at age 30 (A-D), illustrating varying lengths of cardiac transformation of the squamous esophagus resulting from four different levels of cumulative reflux damage (upper frame). This same figure can also show the evolution of cardiac metaplasia over the years in one patient with severe reflux (D).

FIG. 18, provides a schematic diagram illustrating the effect of pH in the pathogenesis of intestinal metaplasia in cardiac mucosa. Four different baseline gastric pHs (1-4) are shown to the right. With reflux, there is a varying gradient of increasing alkalinity to the normal pH of 7 in the esophagus. It is seen that the actual pH at different levels of the esophagus will vary in these situations. If intestinal metaplasia is favored by a pH of 5 (highlighted in a box), the maximal tendency to intestinal metaplasia of cardiac mucosa will occur at different levels in the esophagus. Baseline pH can vary in different people and in the same person with different conditions.

FIG. 19, provides a schematic diagram illustrating the effect of four different pH gradients in the esophagus (A-D). In patient #1 who has a very short segment of cardiac mucosa, intestinal metaplasia (white circle) will occur only in the highest alkaline gradient (D). In patient #2, intestinal metaplasia will occur in gradients C and D only and will extend lower in gradient D than in gradient C. In patient #3, intestinal metaplasia will occur in all four gradients, but it will extend into increasingly distal regions of the esophagus from gradient A to D. In this diagram, intestinal metaplasia occurs only when cardiac mucosa (black) is present.

FIG. 20, provides a schematic diagram illustrating the interplay between different carcinogen doses (A-D) and the level of intestinal metaplasia in the esophagus. Carcinogenesis will not occur unless a sufficient carcinogenic dose reaches an area where there is intestinal metaplasia. In patient #1 (very short segment Barrett esophagus limited to the dilated end-stage esophagus), carcinogenesis is possible in all four dose levels A-D. In patient #2, carcinogenesis will occur only in dose levels A, B and C; it will not occur in the lowest level D. Patient #3 is at risk of cancer only with the highest carcinogen dose environment (A) because it is only at this level that carcinogenic effect reaches the area of intestinal metaplasia proximally in the esophagus.

FIG. 21, provides a schematic diagram illustrating the effect of gastric pH on bile acids. Carcinogenic molecules are produced from bile acids at intermediate levels of gastric pH. Note that bile acids are derived from duodenogastric reflux; entry of bile acids is inevitably associated with alkalinization of gastric juice (B). This natural alkalinization is enhanced in patients receiving acid suppressive drugs (C), shown as increasing carcinogenicity.

FIG. 22, provides a diagram illustrating the distribution of columnar epithelial types in four patients (A, B, C and D) with an identical length of columnar lined esophagus. The amount of intestinal metaplasia (targets for carcinogenesis) is greatest in patient D. Patient A has no intestinal metaplasia and is not at any risk. Patients B and C are at increasing risk. (white=intestinal metaplasia; black=cardiac mucosa; stippled dark gray=oxyntocardiac mucosa).

FIG. 23, provides a photographic illustration of a biopsy of esophagus showing intestinal metaplastic epithelium in the lamina propria under a surface squamous epithelium.

FIG. 24, provides a photographic illustration of a poorly differentiated adenocarcinoma in the lamina propria under an intact surface squamous epithelium.

FIG. 25, provides a schematic diagram illustrating the criteria for “cure” (removal from the reflux-adenocarcinoma sequence) in Barrett esophagus. This may result from conversion of all intestinal and cardiac mucosa to oxyntocardiac mucosa without any change in the length of columnar lined esophagus (upper series); or replacement of columnar epithelium with squamous epithelium. In the latter event, it is very difficult to detect the existence of columnar epithelial elements in the lamina propria under the squamous epithelium.

FIG. 26, provides a graphical illustration of the incidence of adenocarcinoma of the distal esophagus (solid line) and adenocarcinoma of the gastric cardia (dotted line) between 1975 and 2000. Both show an increase; the incidence of adenocarcinoma of the esophagus has increased six-fold during this period.

FIG. 27, provides a flow-chart showing the basic mechanism whereby squamous epithelium is damaged by acid to cause an increased permeability of the epithelium. Other non-acid molecules in the refluxate can then enter the squamous epithelium to interact with the proliferating progenitor cells in the basal region, causing the genetic switch that results in cardiac metaplasia. Points of attack of this process are shown on the right.

FIG. 28, provides a flow-chart illustrating the evolution of cardiac mucosa in one of two different pathways resulting from the presence of exactly opposite factors. Oxyntocardiac mucosa is generated in a high acid, low damage environment; intestinal metaplasia is generated in a less acid (i.e. more alkaline), high damage environment. Only the intestinal metaplastic pathway is susceptible to carcinogenesis.

FIG. 29, provides a schematic diagram illustrating the characteristics of patients with the highest and lowest tendencies to experience a cardiac to intestinal transformation. The patient (#1) with intestinal metaplasia in the shortest segment of columnar lined esophagus has the highest tendency because this requires the greatest alkalinity of gastric juice (D). The patient (#2) with a long segment of columnar lined esophagus devoid of intestinal metaplasia has the lowest tendency because intestinal metaplasia has not occurred in the higher, more alkaline region of the esophagus. On the left, the pH at different levels in the esophagus are shown; if intestinal metaplasia occurs at a pH of 5. the reason for this becomes evident.

FIG. 30, provides a schematic diagram illustrating the characteristics of patients with high and low levels of carcinogenicity of gastric juice. The highest carcinogenicity is in patient #1 who develops cancer in a small area of intestinal metaplasia in the most proximal part of the columnar lined esophagus. The concentration of carcinogen in gastric juice must be very high to reach this target cell. In contrast, patient #2, who has intestinal metaplasia extending all the way down to the gastroesophageal junction is exposed to the highest concentration of carcinogen. If this patient does not progress to cancer, carcinogenicity must be very low.

### DETAILED DESCRIPTION OF THE INVENTION

The current invention is directed to novel methodologies for diagnosing and treating gastroesophageal reflux disease. These methodologies are based on the discovery of a new disease mechanism called hereinafter “reflux carditis”, which is a novel mechanistic view of reflux disease that recognizes cardiac mucosa, which has heretofore been regarded as a normal epithelium in the stomach and an abnormal epithelium in the esophagus, as an abnormal mucosa in both the esophagus and stomach.

Method for Diagnosing Gastroesophageal Reflux

Despite the vast sums of money being spent on research into treating reflux disease, very little progress has been made in preventing the most serious consequence of the disorder—adenocarcinoma. It has been surprisingly discovered that the reason for this continued failure lies in a fundamental misunderstanding of the basic causes of gastroesophageal reflux disease, and the concomitant misdirection of research. Part of the reason for this lack of understanding of the cellular basis of reflux disease is that there is little cross-pollination between the gastroenterologists and surgeons in the field, who have absolutely no training in microscopic pathology, and the pathology establishment. As a result, there is very little specific effort being made to address the approximately 14,000 patients who die every year from esophageal adenocarcinoma.

Although based on a faulty understanding of the mechanism of reflux disease, this failure is directly caused by the misdiagnosis of patients during conventional Barrett esophagus surveillance programs. Accordingly, in a first embodiment, the current invention provides a new method for diagnosing and classifying reflux disease, and thereby preventing reflux-induced adenocarcinoma.

Background

Spechler stated the problem well in 1997: “Conceptually, (columnar lined esophagus) can be defined simply as the condition in which the stratified squamous epithelium of the distal esophagus is replaced by a metaplastic, columnar epithelium. Practically, it can be difficult to apply this conceptual definition in clinical situations because of difficulties in delimiting the distal esophagus and the proximal stomach and in distinguishing normal from metaplastic columnar epithelium on the basis of endoscopic appearance.” (Spechler S J. The columnar lined esophagus. Gastroenterol Clin N Amer 1997:26:455-465, the disclosure of which is incorporated herein by reference.)

In short, the way to define the gastroesophageal junction and distinguish normal from metaplastic columnar epithelium is not by endoscopy. In a first embodiment, the current invention is directed to a method of diagnosing Barrett esophagus using new histologic criteria.

Histologic Definitions of Epithelial Types

Before establishing the histological criteria to be used in the current diagnosis methodology, it is important to have standardized and easily reproducible definitions are used. Moreover, histologic definition of the various types of mucosa is not only easy and reproducible but they can be subject to review because the slides are permanent.

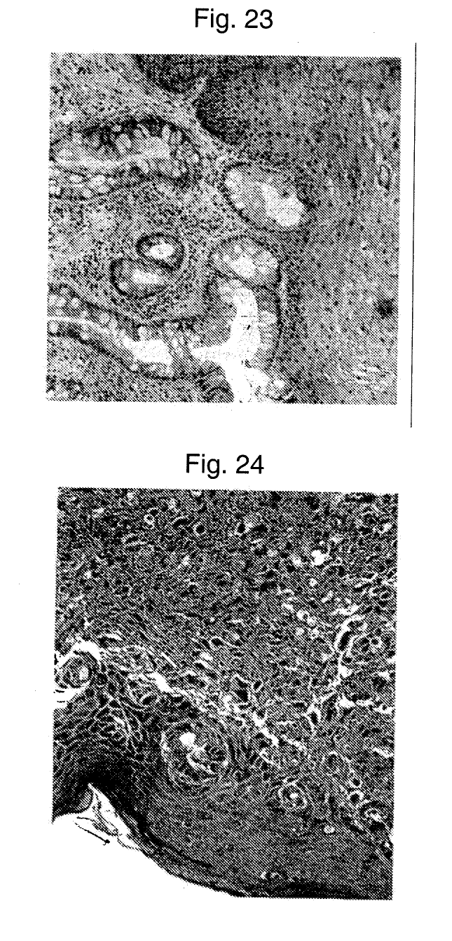

There are only a limited number of epithelial types present in the esophagus and proximal stomach (FIGS. 23-27; Table 1). These were originally defined by Paull et al in 1976 in their classical paper describing the histology of columnar lined esophagus. (Paull A, Trier J S, Dalton M D, Camp R C, Loeb P, Goyal R K. The histologic spectrum of Barrett's esophagus. N Engl J Med 1976; 295:476-480, the disclosure of which is incorporated herein by reference.) These conventional definitions have been modified to better fit current usage, and are summarized in Table 1, below, and in attached (FIGS. 1-5). (Chandrasoma P T, Lokuhetty D M, DeMeester T R, Bremner C G, Peters J H, Oberg S, Groshen S. Definition of histopathologic changes in gastroesophageal reflux disease. Am J Surg Pathol 2000; 24:344-351, the disclosure of which is incorporated herein by reference.)

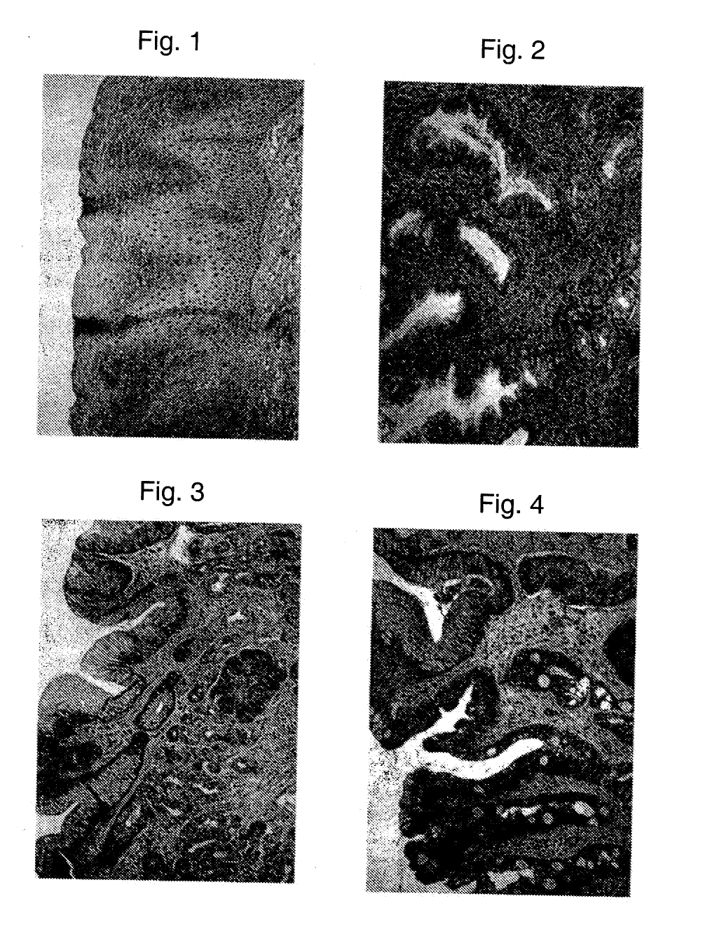

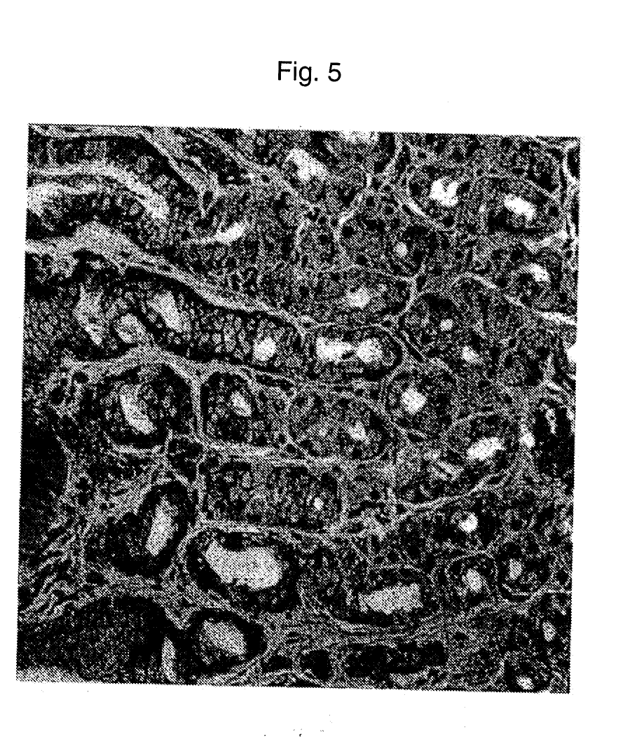

TABLE 1
EPITHELIAL DEFINITIONS

| EPITHELIAL TYPE | DEFINITION |
|---|---|
| Squamous (FIG. 1) | Stratified squamous epithelium |
| Cardiac (FIG. 2) | Columnar epithelium composed only of |
| | mucous cells |
| Oxyntocardiac (FIG. 3) | Columnar epithelium with glands containing |
| | mucous and parietal cells |
| Intestinal (Barrett type) | Goblet cells in cardiac mucosa |
| (FIG. 4) | |
| Intestinal (Gastric type) | Goblet cells in gastric mucosa, either oxyntic |
| | or pyloric |
| Oxyntic (FIG. 5) | Columnar epithelium with glands containing |
| | parietal and chief cells |

Note:
Paull et al's terms for these epithelia are different but the definitions are the same. Cardiac was called junctional, oxynto-cardiac was called fundic, and intestinal was called specialized. Conforming to criteria defining these epithelia is essential. The terms given here are recommended for sake of uniformity.

Apart from the squamous, mucous, parietal, chief and goblet cells that define these epithelial types, one encounters neuroendocrine cells, Paneth cells and serous or pancreatic cells (FIG. 5). These cell types do not influence the basic definition of the epithelial type.

Defining and Testing Normal Anatomy and Histology

The esophagus is a tubular structure that connects the pharynx to the stomach. Normally, it traverses the diaphragmatic hiatus to enter the stomach. By manometry, the distal 4-5 cm of the esophagus is the lower esophageal sphincter; this has an abdominal component that is about 1-2 cm long. (DeMeester T R, Peters J H, Bremner C G, Chandrasoma P. Biology of gastroesophageal reflux disease: Pathophysiology relating to medical and surgical treatment. Ann Rev Med 1999; 50:460-506, the disclosure of which is incorporated herein by reference.) The anatomic position is not a reliable marker of the end of the esophagus; when a sliding hiatal hernia is present, the esophagus is shortened and ends in the thorax. (Allison P R, 1948, previously cited.)

The criteria used to define the gastroesophageal junction at present are: (1) the point of flaring of the tubular esophagus into the saccular stomach, which is the definition recommended for use when esophago-gastrectomy specimens are examined at gross pathology; and (2) the proximal limit of the gastric rugal folds, which is the present widely accepted endoscopic definition. (Association of Directors of Anatomic and Surgical Pathology. Recommendations for reporting of resected esophageal adenocarcinomas. Am J Surg Pathology 2000; 31:1188-1190; Bozymski E M. Barrett's esophagus: Endoscopic characteristics. In Spechler S J, Goyal R K (eds): Barrett's Esophagus: Pathophysiology, Diagnosis, and Management. New York, Elsevier Science Publishing, 1985, pp 113-120; and McClave S A, Boyce H W Jr, Gottfried M R. Early diagnosis of columnar lined esophagus: a new endoscopic criterion. Gastrointest Endosc 1987; 33:413-416, the disclosures of which is incorporated herein by reference.) The rugal folds indicate gastric mucosa. Because gastric mucosa is not damaged by reflux, the proximal limit of the rugal folds remains a constant line as the distal esophagus is altered by the pathologic changes of reflux.

Barrett's 1950 histologic definition that the gastroesophageal junction was the squamo-columnar junction was quickly disproved when columnar lined esophagus was defined. (Allison P R, et al., 1953; and Barret N R, 1957, previously cited.) Thus, while the squamo-columnar junction represents the gastroesophageal junction in a normal person, it clearly moves cephalad in a person whose distal esophagus is damaged by reflux and undergoes columnar metaplasia. (Csendes A, Maluenda F, Braghetto I, et al. Location of the lower esophageal sphincter and the squamous columnar junction in 109 healthy controls and 778 patients with different degrees of endoscopic oesophagitis. Gut 1993; 34:21-27, the disclosure of which is incorporated herein by reference.) It must be recognized that this columnar metaplasia must have a microscopic extent before it becomes grossly or endoscopically visible.

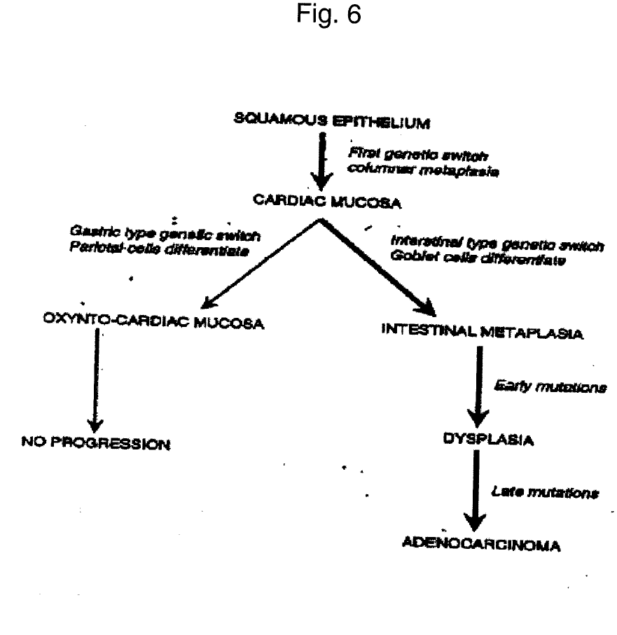

Beginning with Allison, who called the columnar lined esophagus “esophagus lined by gastric mucous membrane,” there has been a tendency to equate the metaplastic esophageal columnar mucosa with gastric mucosa. It is not uncommon even today to find pathologists reporting esophageal biopsies as “gastric or gastric-type mucosa”. (Allison P R, et al., 1953, previously cited.) The current invention is based on the observation that esophageal columnar epithelium is derived from metaplasia of esophageal squamous epithelium; it is not gastric epithelium. In short, it is derived from a genetic switch in the stem cells in the basal layer of the squamous epithelium of the esophagus that causes the cells to differentiate into columnar rather than squamous cells, as shown diagrammatically in (FIG. 6).

The misconception regarding the presence and extent of cardiac mucosa in this region dates back to Allison and Johnstone's original description of columnar lined esophagus, where they conclude that “a variable amount of the oesophagus below the aortic arch may be lined by gastric mucosa of cardiac type”. (Allison P R, et al., 1953, previously cited.) They believed this mucosa to be congenital and therefore a normal variant whatever its length. However, the description of the histology indicates reactive change and the presence of goblet cells. Allison and Johnstone placed this mucosa entirely within the esophagus; they clearly showed that oxyntic mucosa lines the stomach immediately distal to the gastroesophageal junction. A review of Allison's original drawings of this region are worthy of study; he correctly shows squamous epithelium lining the entire normal tubular esophagus. (Allison P R, 1948, previously cited.)

The manner in which the “normal” extent of cardiac mucosa has shrunk and changed from the time of Allison and Johnstone's report of 1953 has been phenomenal. However, these changes were driven largely by opinion, not fact. There were never any formal studies that defined normal histology of this region till Chandrasoma et al reported their autopsy data in 2000. (Chandrasoma P T, Der R, Ma Y, Dalton P, Taira M. Histology of the gastroesophageal junction. An Autopsy study. Am J Surg Pathol 2000; 24:402-409, the disclosure of which is incorporated herein by reference.)

John Hayward, an Australian surgeon, in his classic paper of 1961, vigorously argued against columnar lined esophagus being a normal congenital anomaly and established limits to the acceptable amount of normal cardiac mucosa in the distal esophagus to 1-2 cm of the distal esophagus. (Hayward J. The lower end of the oesophagus. Thorax 16:36-41, the disclosure of which is incorporated herein by reference.) Careful reading of Hayward's report shows that he placed this mucosa entirely in the esophagus. Hayward is oft misquoted in the literature as placing cardiac mucosa in the proximal stomach; while he says that cardiac mucosa “also extends a little way into the stomach” in his opening statement, he explains this later by indicating that this occurs only as a protrusion into the stomach during swallowing. In fact, he says in no uncertain terms: “The stomach should be described as lined by two sorts of epithelium, fundal and pyloric . . . ” and laments that histologists have attributed a gastric location to cardiac mucosa because they mistakenly use the squamo-columnar junction as being equivalent to the end of the esophagus. This same mistake is often repeated in papers even at the present time.

Hayward correctly points out the lack of consistent definition of the term “cardia” and suggests that such a mucous secreting cardiac mucosa was necessary because “if squamous epithelium joined gastric epithelium of fundal (acid-secreting) type directly, it would be liable to digestion at the junction.” This is difficult to understand because the presence of a sphincter mechanism was well recognized both by him and others by this time. (Barrett N R, 1950, previously cited; and Marchand P. The gastroesophageal “sphincter” and the mechanism of regurgitation. Br J Surg 1955; 42:504-513, the disclosure of which is incorporated herein by reference.) The lower esophageal sphincter was known to be an effective barrier that normally prevented esophageal squamous epithelium from being exposed to gastric juice.

From the time of Hayward's report, cardiac mucosa came to be regarded universally as normally lining the distal esophagus. Even today, standard histology textbooks describe the existence of 1-3 cm of “normal” cardiac mucosa in the gastroesophageal region and cardiac mucosa is almost universally believed to line the proximal stomach. Hayward gave no data, either his or from other studies, to support his contention that 1-2 cm of cardiac mucosa was normally found in the distal esophagus. This dogma is not based on fact; and it has now been discovered that when tested, it is not supportable.

There are two available methods of testing the normal extent of cardiac mucosa in the gastroesophageal region:

a) The Demeester Biopsy Protocol

The first is to sample this region in normal people by endoscopic biopsy, following an extensive biopsy protocol, as set forth in Table 2, below. It is not easy to do this because normal people do not easily subject themselves to endoscopic biopsy. All that can be achieved is to biopsy endoscopically normal patients with the clear understanding that endoscopic normalcy does not define normal because there must be a stage of microscopic abnormality that precedes endoscopic abnormality. When these patients also have 24-hr pH testing and complete manometric studies, accurate correlation of histologic data to acid exposure and sphincter abnormalities is possible. Such complete studies are extraordinarily rare in the literature.

TABLE 2
THE DEMEESTER BIOPSY PROTOCOL

| Specimen | Biopsy |
|---|---|
| A | Biopsies of distal stomach: pyloric antrum (3 biopsies) and |
| | gastric body (2 biopsies). |
| B | Biopsies of the proximal limit of the gastric rugal folds with |
| | the endoscope in the retroflexed position (3 biopsies). |
| C | Biopsies (4 quadrant) across the squamo-columnar junction, |
| | attempting to straddle the junction. The distance of this point |
| | from the incisors is stated. |
| D, E, F, | In patients whose squamous epithelium came all the way |
| etc. | down to the proximal limit of the gastric rugal folds (defined |
| | as normal endoscopically), specimens A, B and C are the only |
| | biopsies taken. In patients whose squamo-columnar junction |
| | has visibly moved cephalad, 4 quadrant biopsies are taken |
| | from the endoscopically visualized columnar lined esophagus |
| | at 1 or 2 cm intervals depending on the length of the columnar |
| | lined esophagus. Biopsies from each level are processed as |
| | separate specimens labeled as to the distance from |
| | the incisors. |

b) Autopsy

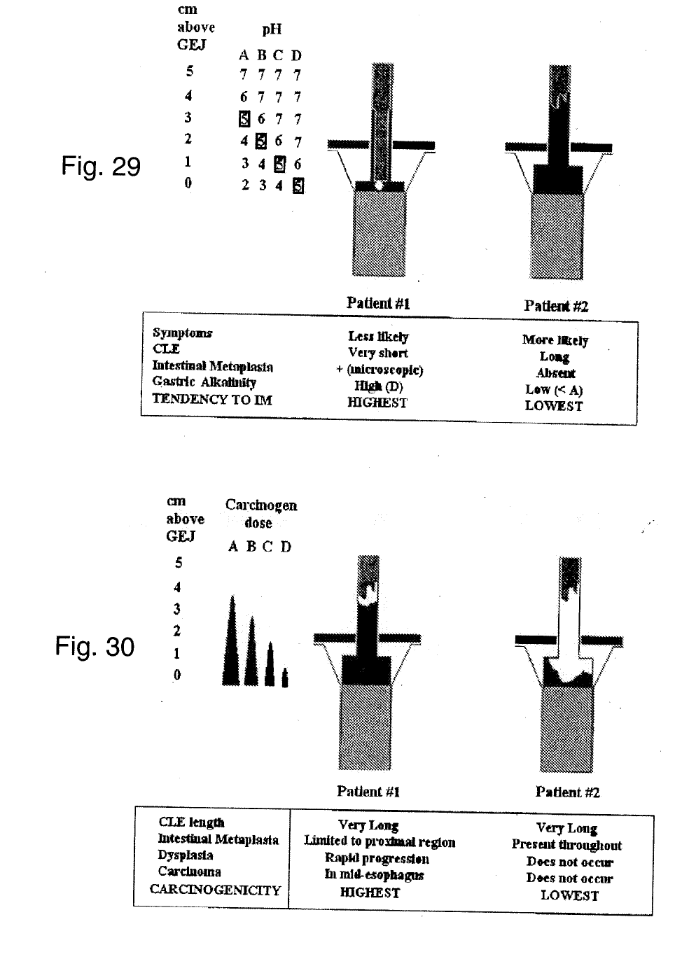

The second method is to study the gastroesophageal junction at autopsy and in esophagectomy specimens. Postmortem autolysis occurs so rapidly in glandular mucosa of this region that it is not a surprise that the twenty-first century dawned before the first autopsy-based histologic study of the gastroesophageal junctional region was published. In a prospective study of 18 autopsies, Chandrasoma et al examined the entire circumference of the gastroesophageal junction, accurately measuring the vertical extent of the various epithelial types. They showed that cardiac mucosa was completely absent in 10/18 (56%) patients with squamous epithelium transitioning directly to parietal cell containing oxynto-cardiac or oxyntic mucosa (FIG. 29). The total cardiac and oxynto-cardiac mucosal length separating squamous and oxyntic mucosa was 0 to 0.8 cm. (Chandrasoma P T, et al., 2000, previously cited.)

While these data have been largely confirmed by autopsy and endoscopic studies reported to date, there is a stubborn and almost defiant defense of the dogma that cardiac mucosa exists as a normal structure. Jain et al, in a study that thoroughly sampled the squamo-columnar junction in endoscopically normal patients, reported the absence of cardiac mucosa in 65% of patients. (Jain R, Aquino D, Harford W V, Lee E, Spechler S J. Cardiac epithelium is fund infrequently in the gastric cardia. Gastroenterology 1998; 114:A160 (Abstract), the disclosure of which is incorporated herein by reference.) Marsman et al., studied 63 patients who had the actual squamo-columnar junction in a single biopsy specimen and reported that “purely cardiac mucosa was present in 39 (62%) biopsies.” (Marsman W A, van Sandick J W, Tytgat G N J, ten Kate F J W, van Lanschot J J B. The presence and mucin histochemistry of cardiac type mucosa at the esophagogastric junction. Am J Gastroenterol 2004; 99:212-217, the disclosure of which is incorporated herein by reference.) Despite finding that cardiac mucosa was absent in 38% of their patients, they inexplicably concluded that “cardiac mucosa was uniformly present adjacent to the squamous epithelium at the EGJ.” Kilgore et al, in the only contradictory autopsy study, reported that cardiac mucosa was present in all their pediatric patients in a length of 0.1-0.4 cm (mean 0.18 cm). (Kilgore S P, Ormsby A H, Gramlich T L, et al. The gastric cardia: fact or fiction? Am J Gastroenterol 2000; 95:921-924, the disclosure of which is incorporated herein by reference.) Review of the methods of this paper shows that these authors used their own unique and therefore incompatible definition of cardiac mucosa. (Chandrasoma P T. RE: Kilgore et al. The gastric cardia: Fact or fiction? (letter). Am J Gastroenterol 2000; 95:2384-2385, the disclosure of which is incorporated herein by reference.)

Critical review of the literature leads to the conclusion that cardiac mucosa is absent in over 50% of the general population. When present, its extent is in the 1-9 mm range in over 95% of the general population and approximately 85% of a population undergoing endoscopy. (Chandrasoma P T, Der R, Ma Y, Peters J, DeMeester T. Histologic classification of patients based on mapping biopsies of the gastroesophageal junction. Am J Surg Pathol 2003; 27:929-936, the disclosure of which is incorporated herein by reference.) In short, no normal structure can be absent in over half of the population and so small in nearly everyone else. Accordingly, the current invention is based on the understanding that cardiac mucosa is not a “native” structure normally present in everyone, and its presence can be used to accurately diagnose gastroesophageal reflux disease.

Significance of Cardiac and Oxynto-Cardiac Mucosa

To prove the hypothesis that cardiac mucosa can be used diagnose gastroesophageal reflux disease, 334 patients were studied; 246 of these had cardiac or oxynto-cardiac mucosa in their biopsies and 88 did not. (Oberg S, Peters J H, DeMeester T R, et al. Inflammation and specialized intestinal metaplasia of cardiac mucosa is a manifestation of gastroesophageal reflux disease. Ann Surg 1997; 226:522-532, the disclosure of which is incorporated herein by reference.) The patients who had cardiac or oxynto-cardiac mucosa were more likely to have abnormal reflux by a 24-hr pH test, and more likely to have abnormalities in their lower esophageal sphincter. This result alone provides some supporting evidence for the basis of the current diagnostic method, namely that cardiac mucosa is an abnormal structure. Even if cardiac mucosa was present in all patients, which it is not, it would not necessarily be a normal structure. Medically, the test of normalcy is whether the presence or absence of the structure is associated with a recognizable abnormality. In this case cardiac mucosa is an abnormal structure whose presence correlates with abnormal reflux.

In a further examination of this question, a group of patients with visible columnar metaplasia of the esophagus was studied. This study demonstrated that the length of metaplastic epithelium showed a highly significant correlation with the severity of reflux. (Chandrasoma P T, et al., 2000, previously cited.) Patients with columnar metaplasia >2 cm in the lower esophagus had significantly more abnormal reflux by 24-hr pH testing than those with <2 cm. Glickman et al in a study of pediatric patients with reflux found that cardiac mucosa was absent in 19% and that patients who had cardiac mucosa exceeding 1 mm in length were more likely to have symptoms of reflux than those who had less than 1 mm. (Glickman J N, Fox V, Antonioli D A, Wang H H, Odze R D. Morphology of the cardia and significance of carditis in pediatric patients. Am J Surg Pathol 2002; 26:1032-1039, the disclosure of which is incorporated herein by reference.) Thus, in one embodiment of the invention the length of cardiac mucosa may be used to provide a directly proportional measure of the amount of reflux, beginning at 1 mm.

Finally, a series of 141 consecutive patients who had cardiac mucosa in their biopsies were also studied. (Der R, Tsao-Wei D D, DeMeester T, et al. Carditis. A manifestation of gastroesophageal reflux disease. 2001; 25:245-252, the disclosure of which is incorporated herein by reference.) All biopsies with cardiac mucosa showed chronic inflammation and reactive changes, even when the immediately adjacent gastric oxyntic mucosa was absolutely normal. The results of this study show that there is a correlation between the severity of chronic inflammation and abnormal reflux by a 24-hr pH test. Thus, the degree of chronic inflammation in cardiac mucosa may be used as a direct measurement of the severity of reflux.

In summary, it has been surprisingly discovered that “histologically normal cardiac mucosa” does not exist; it is always inflamed with histologic evidence of damage and reactive change. Accordingly, the finding of cardiac mucosa in biopsies can be directly correlated with the pathologic diagnosis of “reflux carditis”, which is characterized by chronic inflammation (lymphocytes, plasma cells and frequent eosinophils) and reactive features including foveolar hyperplasia, fibrosis and smooth muscle proliferation. (Chandrasoma P T. Non-neoplastic diseases of the esophagus. In: Chandrasoma P (ed) Gastrointestinal Pathology. Stamford, Conn.: Appleton & Lange, 1999, the disclosure of which is incorporated herein by reference.) Sometimes, this hyperplastic cardiac mucosa produces small polypoid mucosal lesions at the gastroesophageal junction. Data showing that the presence of this epithelium in the junction has also been correlated with an abnormal pH study and therefore justifies attaching the adjective “reflux” to the term “carditis.” The definition of this new diagnosis is detailed in Table 3, below.

TABLE 3
DESCRIPTION OF REFLUX CARDITIS DIAGNOSIS

| | |
|---|---|
| Definition | The finding of cardiac mucosa proximal to gastric oxyntic mucosa. |
| | It is not necessary to say “inflamed cardiac mucosa” because cardiac |
| | mucosa is always inflamed. These biopsies may come to pathologist |
| | labeled at endoscopy as “esophagus”, “gastroesophageal junction”, |
| | “gastric cardia”, “proximal stomach” or “distal to the gastro- |
| | esophageal junction”. The endoscopic landmarks are superceded by |
| | the histologic finding of cardiac mucosa. |
| Histologic | A mucosa containing mucous cells lining the surface, foveolar region |
| Features | and, when present, glands. It shows chronic inflammation (with |
| | eosinophils, plasma cells and lymphocytes in the lamina propria) and |
| | reactive changes (gland distortion, foveolar elongation and serration, |
| | fibrosis and smooth muscle proliferation in the lamina propria) |
| | (FIG. 2) |
| Gross/endoscopic | Usually a flat columnar mucosa between the proximal limit of the |
| appearance | rugal folds and squamous epithelium. In patients who are endo- |
| | scopically normal, it is detectable only by biopsy of the region |
| | immediately distal to the squamous epithelium. When severe, it may |
| | form small polypoid structures. |
| Symptoms | Patients usually have symptoms of reflux. When symptoms are |
| | absent, reflux carditis defines asymptomatic reflux disease. Patients |
| | commonly have an abnormal 24-hr pH study; if the 24-hr pH study is |
| | normal in a patient with reflux carditis, this defines a false negative |
| | 24-hr pH study. |
| Significance | Reflux carditis is the defining diagnostic criterion for reflux disease. |
| | If it is present, the patient has reflux disease at a cellular level. If it |
| | is absent in a patient who has had adequate sampling of the region, |
| | it excludes reflux disease. Being definitive, it has 100% specificity |
| | (always) and 100% sensitivity (except for sampling error). |

When carditis is defined by the histologic criteria given, it can be shown that it is always caused by reflux. Unfortunately, many studies of carditis do not use a histologic definition for the disease. For example, in one of the most influential of studies from the Cleveland Clinic, carditis is defined by the presence of inflammation in a biopsy taken within 5 mm from a normal-appearing squamo-columnar junction; the mucosal type in this biopsy is not mentioned. (Goldblum J R, Vicari J J, Falk G W, et al. Inflammation and intestinal metaplasia of the gastric cardia: the role of gastroesophageal reflux and H. pylori infection. Gastroenterology 1998; 114:633-639; and Goldblum J R. Inflammation and intestinal metaplasia of the gastric cardia: Helicobacter pylori, gastroesophageal reflux disease, or both. Dig Dis 2000; 18:14-19, the disclosures of which are incorporated herein by reference.)

This is not carditis as defined histologically; rather inflammation is being studied at an anatomically defined point in the gastric cardia which is then defined as “the most proximal portion of the stomach occupying a small zone immediately distal to the esophagogastric junction.” (Goldblum J R, et al., 1998; and Goldblum J R, 2000, the disclosures of which are incorporated herein by reference.) The mucosa at this point 0.5 cm from the squamo-columnar junction has a high likelihood of being oxyntic mucosa based on their own autopsy study that reported the mean length of cardiac mucosa to be 0.18 cm. (Kilgore S P, et al., 2000, previously cited.) Inflammation of oxyntic mucosa in the proximal stomach or “gastric cardia” is not carditis, it is gastritis. Accordingly, the conclusion of the study, that gastritis is caused by Helicobacter pylori and not reflux disease, is self-evident.

Studies that define carditis by the histologic criterion of an inflamed cardiac mucosa find a strong association with reflux. (Oberg, et al., 1997; Der R., et al., 2001; Clark G W, Ireland A P, Chandrasoma P, et al. Inflammation and metaplasia in the transitional epithelium of the gastroesophageal junction: a new marker for gastroesophageal reflux disease. Gastroenterology 1994; 106:A63 (Abstract); Riddell R H. The biopsy diagnosis of gastroesophageal reflux disease, “carditis” and Barrett's esophagus, and sequelae of therapy (review). Am J Surg Pathol 1996; 20 (suppl):S31-50; Lembo T, Ippoliti A F, Ramers C, Weinstein W M. Inflammation of the gastroesophageal junction (carditis) in patients with symptomatic gastro-esophageal reflux disease: a prospective study. Gut 1999; 45:484-488; and Bowrey D J, Clark G W B, Williams G T. Patterns of gastritis in patients with gastro-oesophageal reflux disease. Gut 1999; 45:798-803, the disclosures of which are incorporated herein by reference.) Studies that define carditis as “inflammation in a biopsy taken from the anatomic gastric cardia” frequently find an association with Helicobacter pylori, or both Helicobacter pylori and reflux. (Goldblum J R, et al., 1998; Goldblum J R, 2000; Genta R M, Huberman R M, Graham D Y. The gastric cardia in Helicobacter pylori infection. Hum Pathol 1994; 25:915-919; and Hackelsberger A, Gunther T, Schultze V, et al. Prevalence and pattern of Helicobacter pylori gastritis and the gastric cardia. Am J Gastroenterol 1997; 92:2220-2223, the disclosures of which are incorporated herein by reference.) In short, when defined by strict histologic criteria, as proposed in the current invention, carditis is always reflux carditis.

The simultaneous occurrence of gastroesophageal reflux disease and Helicobacter pylori gastritis in the same patient leads to confusion in the unwary. Both are common diseases and it is to be expected that they may occur simultaneously. Helicobacter pylori causes gastritis that favors the antrum but frequently causes a pangastritis. In patients who have no reflux-induced cardiac mucosa, pangastritis produces inflammation of the entire gastric mucosa, extending all the way proximally to the normal squamo-columnar junction. When coexisting reflux disease has produced metaplastic cardiac mucosa in the lower esophagus, the Helicobacter pylori infection extends into the metaplastic cardiac mucosa in the esophagus. This results in an aggravation of the severity of chronic inflammation in cardiac mucosa and adds a component of acute inflammation. The exacerbation of inflammation of reflux carditis when it becomes secondarily infected by Helicobacter pylori is confusing unless the pathology is properly interpreted.

Histologic Diagnosis of Reflux

Once it is observed that cardiac mucosa does not exist normally in either the esophagus or the stomach, the understanding and diagnosis of reflux disease becomes trivial. The first result it the observation that only two epithelial types are normal: esophageal squamous epithelium and gastric oxyntic mucosa. In patients suffering from gastroesophageal reflux the squamous epithelium of the esophagus is damaged, undergoing metaplasia to columnar epithelium. In contrast, gastric oxyntic mucosa is designed to withstand gastric acid.

Based on the recognition of this normal histology, accurate histologic definitions now become feasible and self-evident, and are set forth in Table 4, below.

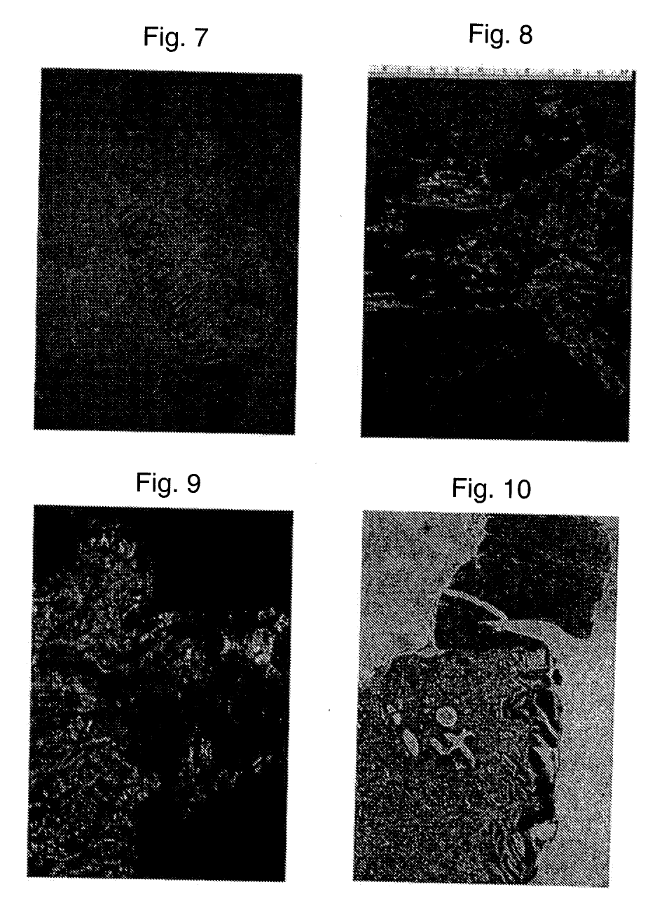

TABLE 4
HISTOLOGIC DEFINITIONS OF ANATOMY AND HISTOLOGY

| | |
|---|---|
| The Esophagus: | The esophagus is that part of the foregut that is lined |
| | by squamous epithelium or metaplastic columnar |
| | epithelia that have resulted from the effect of reflux on |
| | the squamous epithelium (FIGS. 7-9). |
| The Stomach: | The stomach is that part of the foregut that is lined by |
| | gastric oxyntic mucosa. It is unaffected by reflux and |
| | its position remains constant. At endoscopy and gross |
| | examination, the best indicator of gastric oxyntic |
| | mucosa is the presence of rugal folds but histology is |
| | the ultimate defining modality. |
| Gastroesophageal | This is the proximal limit of gastric oxyntic mucosa. In |
| Junction: | normal patients, the squamous epithelium is |
| | coincidental with this point (FIG. 10). In patients with |
| | reflux damage, the squamous epithelium undergoes |
| | columnar metaplasia, causing the squamocolumnar |
| | junction to move cephalad (FIGS. 7-9). |
| Normal Columnar | The only normal columnar epithelium is gastric |
| Epithelium: | oxyntic mucosa (FIG. 10). |
| Columnar Lined | The segment of distal esophagus that is interposed |
| Esophagus: | between the gastroesophageal junction and squamo- |
| | columnar junction as the squamous epithelium |
| | undergoes reflux induced metaplasia (FIGS. 7-9). |
| Metaplastic | Cardiac mucosa with and without intestinal metaplasia, |
| Esophageal | oxynto-cardiac mucosa (FIGS. 7-9). These epithelia |
| Columnar | are always esophageal, irrespective of any endoscopic |
| Epithelium: | opinion. |

Using these highly reproducible histologic definitions, the current invention provides a technique for recognizing and diagnosing both the normal condition (where gastric oxyntic mucosa is the only normal columnar epithelium, as shown in FIG. 10), and increasing degrees of reflux damaged esophagus, measured primary by the amount of squamous epithelium that has undergone columnar metaplasia (recognized by the presence of cardiac mucosa with and without intestinal metaplasia and oxynto-cardiac mucosa, as shown in FIG. 7, and gross separation of the squamo-columnar junction from the rugal folds, as shown in FIGS. 8 and 9). (Chandrasoma P. Pathophysiology of Barrett's esophagus. Semin Thorac Cardiovasc Surg 1997; 9:270-278, the disclosure of which is incorporated herein by reference.)

This diagnostic technique is summarized in Table 5, below.

TABLE 5
CRITERIA TO DIAGNOSE REFLUX

| | |
|---|---|
| No evidence of | These patients have a normal esophagus completely |
| reflux-induced | lined by squamous epithelium and a normal stomach |
| damage: | completely lined by gastric oxyntic mucosa (FIG. 10). |
| | These patients usually have no symptoms of reflux, |
| | have a normal 24-hr pH test and are endoscopically |
| | normal. |
| Mild reflux: | These patients have a microscopic segment of |
| | metaplastic columnar epithelium between the |
| | squamous epithelium and gastric oxyntic mucosa |
| | (FIG. 7). They may have symptoms and often have |
| | a slightly abnormal 24-hr pH test. They have no |
| | visible endoscopic abnormality. They can be |
| | diagnosed only by biopsy. These patients are not |
| | usually biopsied in clinical practice. |
| Moderate reflux: | These patients have a visible but short (<2 cm) |
| | segment of endoscopically recognizable metaplastic |
| | columnar epithelium between the squamous |
| | epithelium and gastric oxyntic mucosa (FIG. 8). This |
| | may be circumferential or appear as tongues of |
| | metaplastic epithelium moving into the squamous |
| | epithelium, causing serration of the Z line. |
| Severe reflux: | These patients have a visible long segment of |
| | metaplastic columnar lined esophagus (>2 cm), |
| | usually circumferential with an irregular upper border |
| | (FIG. 9). They are usually symptomatic and have an |
| | abnormal 24-hr pH test. |

In short, in one embodiment the current invention uses the presence of metaplastic columnar epithelium to diagnose reflux disease, and uses its length to define the severity of reflux disease. It is also shown that such a method is superior to any conventional measure. Moreover, because the main complication of reflux disease is adenocarcinoma, it makes good sense to define this disease in terms of the metaplastic columnar epithelium it causes rather than the residual squamous epithelium. Presently used grading systems for reflux such as the Savary-Miller and Los Angeles classifications assess changes in the squamous epithelium; these are useful in assessing how erosive reflux esophagitis responds to treatment, but are irrelevant in predicting adenocarcinoma risk, as further described below.

Method for Diagnosing Adenocarcinoma

To understand the basis of the new methodology for diagnosing patients at high risk for adenocarcinoma one must first have an understanding in the mechanisms of the cellular changes that occur during reflux disease. Ultimately, every change must be explained on the basis of a cellular interaction between a cell that is present in the patient and a molecule or combination of molecules in the refluxate. Such interactions may be classified be of two kinds:
(a) Destructive or injurious where a molecule causes cell damage by a chemical reaction with cell components. This is the obvious mechanism in high dosage radiation and caustic damage where the physical agent directly causes cell necrosis on contact because of some lethal interaction between the cell (probably cell wall) and the molecule. Acid in high enough concentration has the potential to induce direct cell damage in all types of esophageal epithelia, whether squamous or metaplastic columnar.
(b) Interactive, where the molecule in the refluxate combines with a cell component, usually a receptor to induce a change in the structure and/or function of the cell. The esophageal epithelia, both squamous and metaplastic columnar, have a predominantly non-secretory function, although mucous glands, pancreatic cells, Paneth cells and parietal cells are capable of secretion. The important clinical result of molecule-cell interactions are alterations in genetic control of the cell that result in different metaplastic pathways and carcinogenesis. Interactive mechanisms generally preserve the altered cell in contrast to the destructive influences which damage and destroy the cells.

There is a broad difference in the type of agent most likely to produce these types of cellular change. Destructive injuries tend to be the mechanism of physical agents and simple chemicals such as highly ionic compounds (the coagulative heat of a laser, the hydroxyl ions of lye, the free radicals generated by radiation, and the free hydrogen ions of acid). Ionic agents are harmless until they reach a sufficient concentration where they cause direct damage of the cell membrane or enter the cells, altering pH that may induce changes in chemical reactions in the cell. For example, it is very unlikely that any cell has receptors to simple ionic compounds. Cell receptors are generally complex and interact with larger molecules. Accordingly, larger molecules such as complex nitrogenous compounds and bile salt derivates are in most cases likely responsible for interactive changes.

In summary, the cellular events that occur when gastroesophageal reflux occurs are the result of the interaction of the cells that are exposed and the nature of the refluxate. A summary of these events and interactions are summarized in Table 6, below, which provides a classification of cellular changes of gastroesophageal reflux disease by probability of damage mechanism or an interactive change caused by a molecule in the refluxate combining with a receptor on the surface of a target cell.

TABLE 6
CLASSIFICATION OF CELLULAR CHANGES OF REFLUX
DISEASE

| Injurious/Destructive Phenomena | Agent Involved |
|---|---|
| Increased squamous epithelial cell loss and reactive | Acid |
| basal cell hyperplasia. | |
| Squamous epithelial inflammation (reflux esophagitis) | Acid |
| Chemoattraction of intraepithelial eosinophils | Acid |
| Squamous epithelial erosion/ulceration (erosive | Acid |
| esophagitis) | |
| Dilated intercellular spaces - increased squamous | Acid |
| epithelial permeability | |
| Cardiac epithelial inflammation, cell loss and reactive | Acid/others |
| proliferation | |
| Intestinal epithelial inflammation, cell loss and | Acid/others |
| proliferation | |
| Interactions Between Cells And Molecules | Agent Involved |
| Gene (e.g., Cox-2) activation in squamous | Unknown |
| epithelium | |
| Cardiac metaplasia of squamous epithelium | Unknown |
| Parietal cell induction in cardiac mucosa | Unknown/Acid |
| (oxyntocardiac mucosa) | |
| Goblet cell induction in cardiac mucosa (intestinal | Unknown/bile |
| metaplasia) | salts |
| Carcinogenesis in intestinal metaplasia | Unknown/bile |
| | salts |

As summarized in the above table, acid is very likely the main damage inducing molecule in the refluxate, although evidence exists for significant damage-producing capability in other molecules in the refluxate such as bile salt components. Acid is responsible for numerous changes in the squamous epithelial cells, ultimately causing increased permeability that allows refluxate molecules of all types to gain access to the proliferative stem cell pool in the basal region of the epithelium. (Tobey N A, Carson J L, Alkiek R A, et al. Dilated intercellular spaces: A morphological feature of acid reflux-damaged human esophageal epithelium. Gastroenterology 1996; 111:1200-1205; and Tobey N A, Hosseini S S, Argore C M, Dobrucali A M, Awayda M S, Orlando R C. Dilated intercellular spaces and shunt permeability in non-erosive acid-damaged esophageal epithelium. Am J Gastroenterol 2004; 99:13-22, the disclosures of which are incorporated herein by reference.) Acid is therefore the key that unlocks the squamous epithelial barrier and permits other luminal molecules in the refluxate to gain access to the proliferative stem cell pool. Acid damage sets the stage for molecular interactions with cell surface receptors causing metaplasia to different types of columnar epithelia and inducing genetic changes in the cells that cause progression in the reflux to adenocarcinoma sequence.

Mechanism of Metaplasia

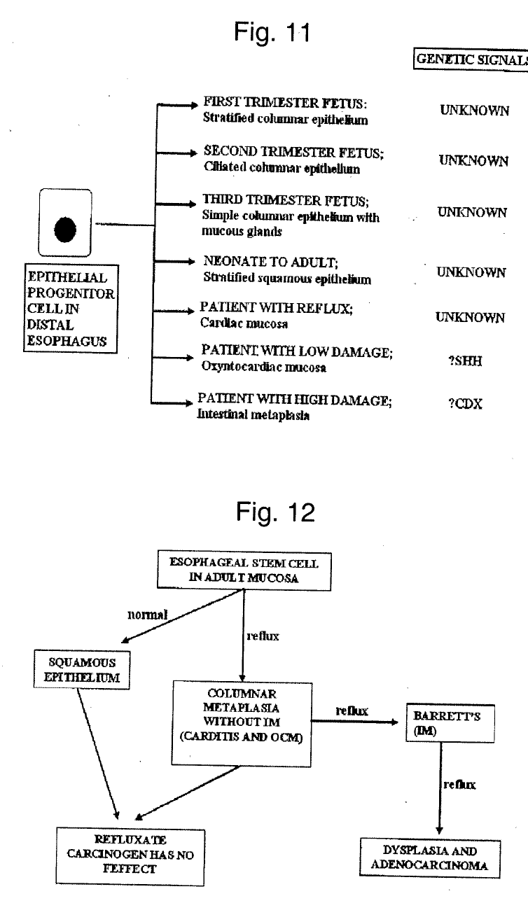

The progenitor stem cell in any given location of the gastrointestinal tract is responsible for the type of epithelium that is present at that site. In turn, the type of epithelium that is generated depends on the genetic signals active in that cell that direct differentiation. In the esophagus, the differentiating signal develops via multiple fetal signals until the final adult genetic signal is established in early postnatal life that directs normal differentiation into stratified squamous epithelium (See, e.g., FIG. 11). All fetal esophageal genetic signals that direct columnar epithelial cell differentiation are suppressed. Genetic signals that direct epithelial differentiation in other parts of the gastrointestinal tract are not active at any point in normal development of the esophageal epithelial progenitor cell (except an intestinal signal which may rarely be transiently expressed in the fetus). These signals remain within the genome of the esophageal epithelial cell in a suppressed state.

Metaplasia is a process that is simply a change in the genetic signal of the progenitor cell to direct the formation of a particular epithelium, as shown schematically in FIG. 11. This does not involve “movement” of one epithelial type to another location. Thus, columnar metaplasia of the esophagus is caused by an alteration of the esophageal epithelial progenitor cell's genetic signal that causes it to differentiate into columnar rather than squamous epithelium. Different types of columnar epithelia are produced from activation of different genetic signals that direct cell differentiation. Columnar metaplasia is not the replacement of esophageal epithelium by “gastric epithelium moving up into the esophagus”.

The only situation where there is lateral movement of progenitor cells is when erosion and ulceration occur; in these cases the progenitor cells move into the denuded area either from the adjacent epithelium, from esophageal gland ducts, or even from circulating stem cells. Even in these cases, however, it is likely that when the progenitor cell reaches its new location it takes on the behavior and differentiation inherent to the new location. Circulating stem cells populating the esophagus do not differentiate into hepatocytes or hematopoietic cells. As a result, Applicants propose that it is a misconception that the columnar lined esophagus is composed of gastric mucosa. This concept was introduced by Allison and Johnstone in the first description of the entity in 1953, and as previously discussed still persists in the minds of many people, and continues to cause misdiagnosis and poorly designed treatment regimes.

With the genetic process described in FIG. 11 in mind, attention can now be turned to the mechanism of metaplasia. Under this regime, it is proposed that metaplasia might results from the activation of suppressed genetic signals of one of two types: (a) Fetal esophageal epithelial signals, and (b) Epithelial signals that are aberrant for the esophagus and normally seen in other parts of the gastrointestinal tract.

If metaplasia results from fetal esophageal epithelial signals, it will result in epithelia that were seen transiently during fetal life during development. Cardiac mucosa is one such epithelium. Cardiac mucosa resembles the non-ciliated columnar epithelium that is present in the third trimester at the distal end of the esophagus. This may consist of a flat epithelium, a columnar epithelium with rudimentary foveolar pits or one with mucosal glands composed only of mucous cells.

In contrast, if metaplasia results from epithelial signals that are aberrant for the esophagus and normally seen in other parts of the gastrointestinal tract, it will produce columnar epithelia that are not normally seen in fetal esophagus during development. These would include the gastric differentiating signal that directs the formation of parietal cells; this is activated in certain conditions and only when cardiac mucosal metaplasia is present in the esophagus. Oxyntocardiac mucosa does not arise directly from squamous epithelium, instead there is evidence that the Sonic Hedgehog gene system is involved in gastric-type differentiation.

Finally, another potential differentiating signal is the intestinal signal which directs the formation of goblet cells similar to those in the intestine. This is activated in certain conditions and only when cardiac mucosal metaplasia is present in the esophagus. Intestinal metaplasia does not arise directly from squamous epithelium. There is evidence that the CdX gene systems are involved in this intestinal-type differentiation (FIG. 11).

In one embodiment, the treatment and diagnosis methodology of the current invention is based on the discovery that the only esophageal epithelial type on which carcinogens in the refluxate act is the intestinal type, as shown schematically in (FIG. 12). In summary, it is proposed that when the genetic switch that directs intestinal metaplasia occurs, there is a transformation of the esophageal epithelial cell that makes the cell susceptible to the action of carcinogens.

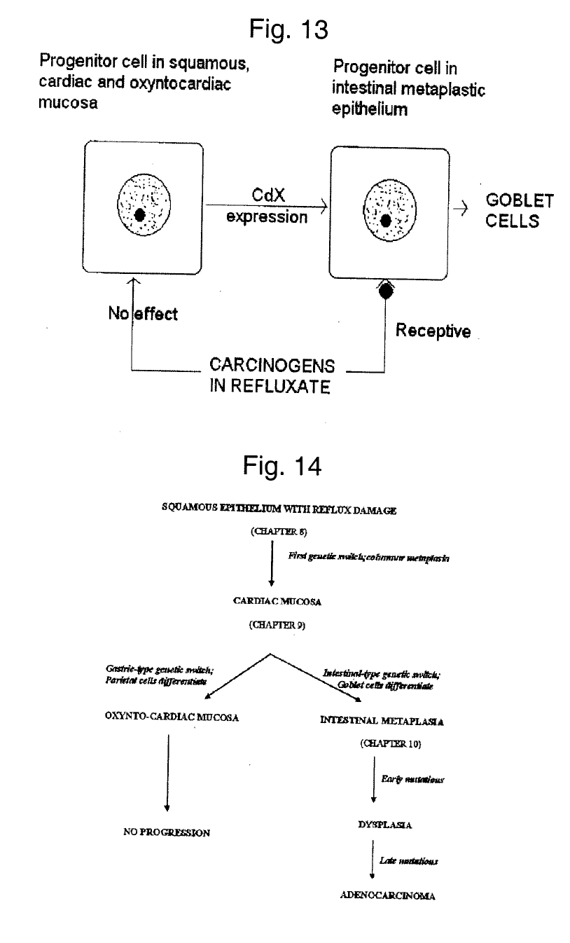

Accordingly, as will be discussed further later, in one embodiment of the current invention it is proposed that the configuration of the cell receptor may be altered to make the cell capable of a new interaction with a carcinogenic molecule (FIG. 13).

Mechanism of Reflux-Adenocarcinoma

The early part of the reflux-adenocarcinoma sequence that takes squamous epithelium to intestinal metaplasia results from activation of normally suppressed genes that are involved in directing differentiating in the gastrointestinal tract (“genetic switches”). These are simple differentiating signals and merely lead to the formation of an orderly adult epithelium that is different to the native squamous epithelium of the esophagus. Accordingly, in one embodiment of the current invention a treatment methodology is proposed that would reverse these genetic switches through therapeutic intervention.

In contrast, the genetic changes that convert intestinal metaplasia to dysplasia and adenocarcinoma would seem to be irreversible and abnormal genetic mutations. These mutations that are the essence of carcinogenesis give the affected cells a growth advantage, lack of orderliness in proliferation, and other characteristics that characterize the uncontrolled growth of neoplasia. As such, these mutations are difficult to reverse and less likely to be easily amenable to a genetic therapeutic intervention.

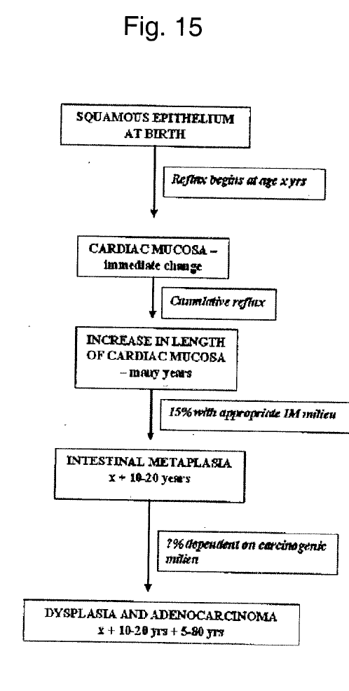

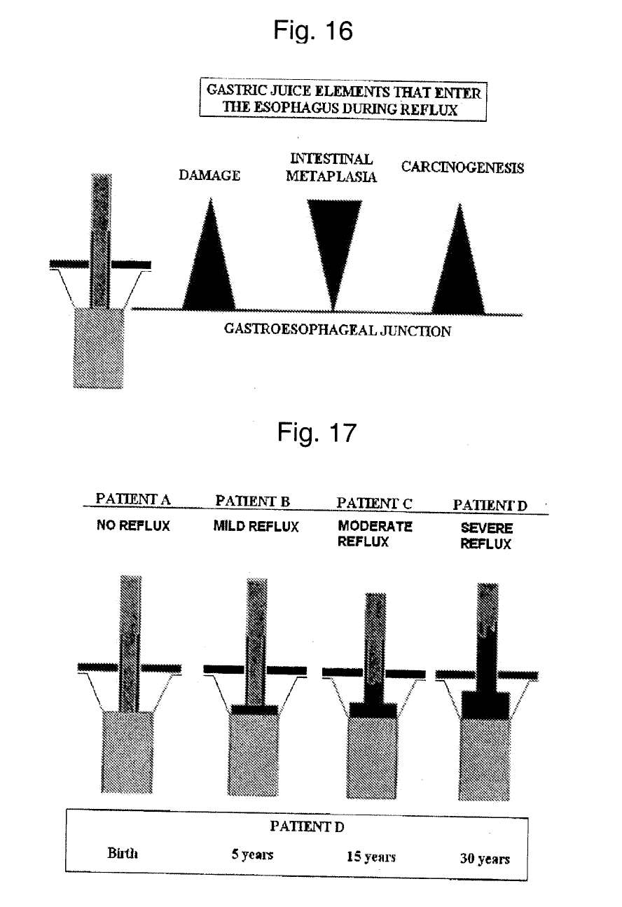

The cellular changes that occur in the reflux-adenocarcinoma sequence are show schematically in (FIG. 14). The patient passes through this sequential change very slowly, over many years, as shown in (FIG. 15). In terms of a treatment methodology this means that there is more than sufficient time to effect a treatment. The actual speed and quantity of change in any patient as they progress to higher risk stages in the reflux-carcinoma sequence is dependent on three critical and independent events, as shown schematically in (FIG. 16), and discussed in further detail below.

(a) The Severity of Damage to Squamous Epithelium:

1. The severity of damage is generally dependent on the amount of reflux and the age of its onset, and is usually acid-dependent; it is maximal at the gastroesophageal junction where acid exposure is greatest, as shown in (FIG. 16). Damage of this type converts squamous epithelium to increasing lengths of cardiac mucosa. Cameron et al showed that the final metaplastic length of squamous epithelium is established at a young age in most patients. (Cameron A J, Lomboy C T. Barrett's esophagus: age, prevalence, and extent of columnar epithelium. Gastroenterology. 1992; 103:1241-5, the disclosure of which is incorporated herein by reference.) One can reasonably assume that in most people, reflux begins fairly early in life and continues to cause increasing cardiac metaplasia of esophageal squamous epithelium for 10-20 years, finally reaching a state of equilibrium where the separation of squamous epithelium from the point of reflux (the gastroesophageal junction) is adequate to prevent further damage. At this stage, squamous epithelial damage stops sufficiently to prevent further cardiac metaplasia and the columnar lined esophagus becomes stable in length (FIG. 17). The length of columnar lined esophagus is a testament to the cumulative lifetime cellular damage that has occurred in the esophagus as a result of chronic gastroesophageal reflux. The current invention proposes new treatments to address this damage, damage that remains untreated in current treatment regimes as evidenced by the frequent presence of long segments of columnar lined esophagus found from the 1930-1960 era in treated patients.

In many patients with mild reflux, all cardiac mucosa transforms to oxyntocardiac mucosa; this is a natural “cure” (i.e., removal from the reflux-adenocarcinoma sequence) that has been termed compensated reflux because these patients are not at risk for intestinal metaplasia or carcinoma. These “compensated” patients make up between 55-65% of the population that have only oxyntocardiac mucosa between the squamous epithelium and gastric oxyntic mucosa, as confirmed by both autopsy populations (Chandrasoma et al 2000; and Zhou H, Greco M A, Daum F, et al. Origin of cardiac mucosa: ontogenic considerations. Ped Dev Pathol 2001; 4:358-363, the disclosures of which are incorporated herein by reference), and clinical populations when otherwise “normal” people are biopsied. (Marsman W A, van Sandyck J W, Tytgat G N J, ten Kate F J W, van Lanschot J J B. The presence and mucin histochemistry of cardiac type mucosa at the esophagogastric junction. Am J Gastroenterol 2004; 99:212-217, the disclosure of which is incorporated herein by reference.)

(b) The Tendency to Intestinal Metaplasia:

The cardiac mucosa generated from squamous epithelium by reflux evolves into oxyntocardiac and intestinal metaplasia based on the factors that have been discussed above. This results in an infinitely variable mixture of the three epithelial types in columnar lined esophagus.

Unlike damage, the tendency for intestinal metaplasia is greater in the more proximal esophagus than at the gastroesophageal junction as shown in (FIG. 16). In a given segment of cardiac metaplasia, intestinal metaplasia almost always occurs in the most proximal region, immediately distal to the squamocolumnar junction. The prevalence of intestinal metaplasia in columnar lined esophagus increases as the length of columnar lined esophagus increases. The current inventive methodology proposes that intestinal metaplasia of cardiac mucosa is a molecular change that is pH dependent, and that it is promoted by a reduction in acidity, i.e. an increase in pH.

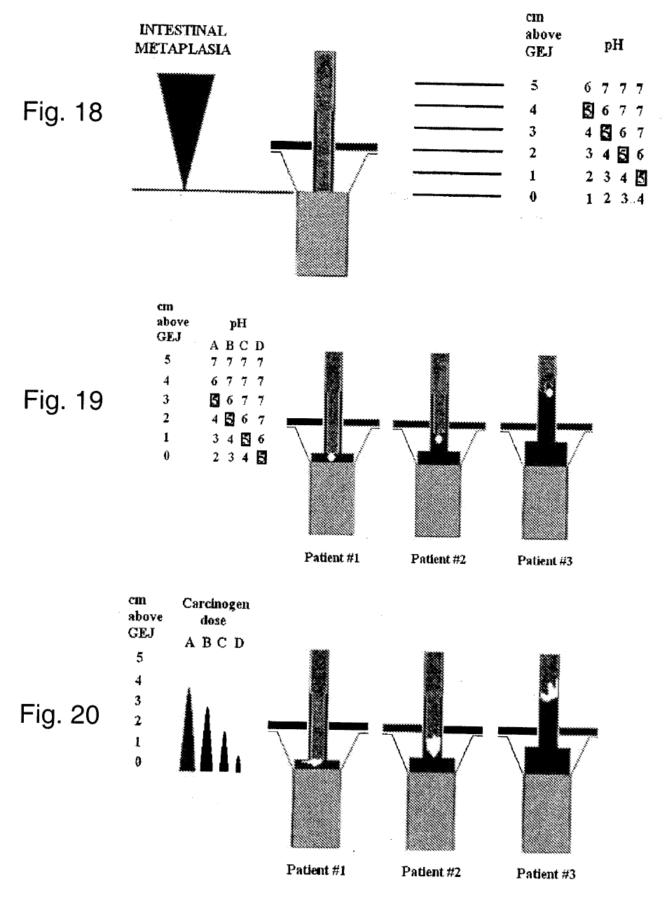

The pH at the gastroesophageal junction is the baseline gastric pH. In the normal patient without reflux, esophageal pH is neutral (i.e. 7). When reflux occurs, the pH in the esophagus has been shown to develop a gradient from low at the gastroesophageal junction to 7 in the proximal esophagus, as shown in (FIG. 18). In patients who have alkaline (i.e. pH >7) gastric juice, reflux can render the pH in the esophagus alkaline and the gradient is reversed, but this is a rare occurrence. In FIG. 18, it is assumed that intestinal metaplasia of cardiac mucosa occurs in a given patient at a pH of 5. It is shown that the point in the esophagus where this pH is reached will vary with the baseline gastric pH. As the gastric juice becomes more alkaline the critical point in the esophagus where intestinal metaplasia is generated becomes more distal. The natural alkalinizer of gastric juice is duodenogastric reflux. However, the use of acid suppressive drugs adds to this natural alkalinizing effect and will tend to lower the critical point at which intestinal metaplasia occurs in the esophagus.

When one superimposes the damage element to this equation, as shown in (FIG. 19), it is seen that a given patient has cardiac mucosa in the esophagus to a point above the gastroesophageal junction that depends on acid exposure of the squamous epithelium which is almost opposite to that which causes intestinal metaplasia. Intestinal metaplasia will occur only if the patient has cardiac mucosa at the critical point. With longer segments of cardiac mucosa in the esophagus, the degree of gastric juice alkalinization needed to cause intestinal metaplasia decreases (FIG. 19). This serves to explain why intestinal metaplasia occurs in the most proximal region of cardiac mucosa and why the prevalence of intestinal metaplasia increases as the length of cardiac mucosa increases.

There has been an historical increase in both the prevalence of intestinal metaplasia within columnar lined esophagus and the amount of intestinal metaplasia in columnar lined esophagus. It is proposed that this increase can be explained entirely by a decrease in the baseline acidity of gastric juice. It is probable that much of this alkalinity is natural, although it is difficult to find evidence for or a reason for a historic increase in duodenogastric reflux. However, there is no question that gastric acidity is reduced in patients who are treated with acid suppressive medications. With all other things being equal it is proposed that acid suppression will tilt the equilibrium to (i) induce intestinal metaplasia in cardiac mucosa of shorter length and (ii) increase the amount of intestinal metaplasia that occurs in columnar lined esophagus (FIG. 19).

Intestinal metaplasia still occurs only in a minority of patients with cardiac mucosa, usually after many years following the initial cardiac metaplasia of squamous epithelium. It is uncommon for intestinal metaplasia to occur before 20 years of age and the median age at which intestinal metaplasia (Barrett esophagus) is detected is around 40 years. The significant time interval between the occurrence of cardiac mucosa and intestinal metaplasia is a window of opportunity for therapeutic interventions to prevent intestinal metaplasia that have heretofore received scant attention because of the belief that cardiac mucosa is a normal epithelium.

(c) Carcinogenesis in Intestinal Metaplasia:

The risk of cancer begins only in those patients who develop intestinal metaplasia. Non-intestinalized columnar metaplastic epithelium and squamous epithelium of the esophagus are not believed to be directly susceptible to carcinogenesis, as discussed previously in (FIG. 12).

It is proposed herein that carcinogenesis in intestinal metaplasia is caused by multiple interactions or “hits” whereby the susceptible target cell in intestinal metaplastic epithelium is acted upon by a carcinogenic molecule/s in the gastric refluxate. This interaction is dependent on: (i) the presence of the target cell, which is reliably indicated by the presence of intestinal metaplasia in the columnar lined segment; (ii) the presence of the carcinogen, which is unknown, but possibly a derivative of bile acid metabolism; and (iii) the ability of the carcinogenic molecule to access the target cell.

When these three essential elements are present, carcinogenesis is promoted by increased mitotic activity in the target cell which increases the likelihood of mutagenic events and probably the concentration of the carcinogenic molecule, assuming that the molecule induces a dose-related effect which is very likely. Because the carcinogen is in gastric juice, the maximum concentration of carcinogen is at the gastroesophageal junction. Without reflux, the carcinogen never reaches the esophagus and cancer does not occur. With reflux, the carcinogen enters the esophagus. Based on the fact that reflux has an upward propulsion into the esophagus followed by a clearing mechanism, the exposure of the esophagus to carcinogen is maximal just above the gastroesophageal junction with a decreasing gradient as one moves proximally in the esophagus (FIG. 20). The exact height to which effective carcinogen dose is delivered by reflux will depend on the severity of the reflux and the concentration of carcinogen, as shown in (FIG. 20).

As has been discussed, the maximum carcinogenic tendency operates in the lowest regions of the esophagus. This is almost exactly opposite to the tendency to intestinal metaplasia. Patients with intestinal metaplasia limited to the most proximal region of a long segment of columnar lined esophagus are protected because the likelihood that the carcinogen will reach an effective dose level so high in the esophagus is low (FIG. 20). The risk, however, increases as the intestinal metaplasia moves distally in such a patient. The patient who develops intestinal metaplasia in very short segments of columnar lined esophagus has the highest theoretical exposure to the carcinogen because of the proximity to gastric juice (FIG. 20). This is counterbalanced by the fact the number of target cells in longer segments is greater, resulting in a complex interplay of factors. There is clinical evidence that provides supporting evidence for this hypothesis. Specifically, adenocarcinomas in Barrett esophagus much more commonly occur in the distal part of the esophagus than in the proximal esophagus even when long segments of Barrett esophagus are present.

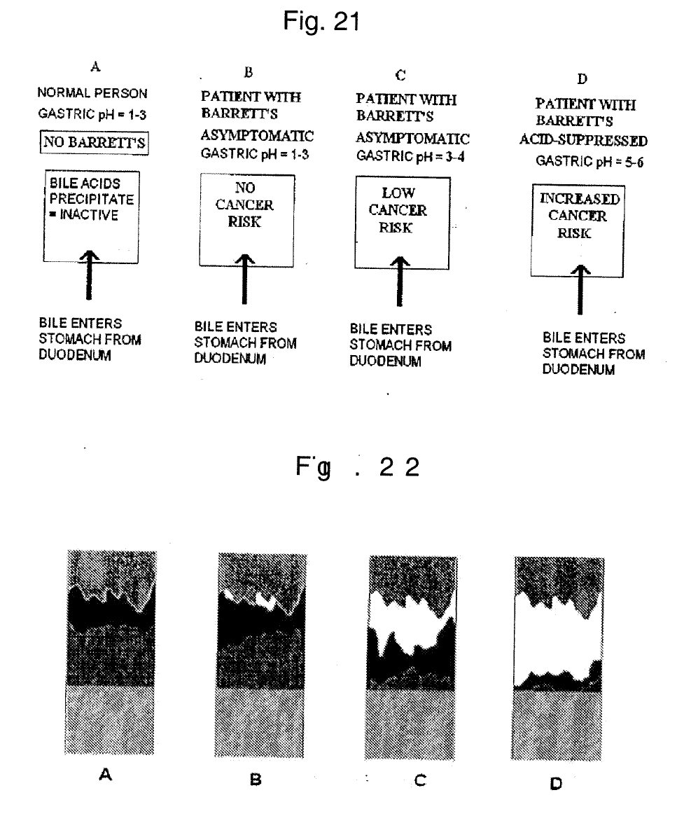

This interplay between the tendency to intestinal metaplasia in cardiac mucosa and carcinogenesis can also explain the historic increase in adenocarcinoma. There is evidence that alkalinization of gastric juice by duodenogastric reflux promotes the conversion of bile acid to carcinogenic metabolites at pH ranges that are intermediate between normal gastric juice (pH 1-3) and normal duodenum (pH 6-7), as demonstrated and discussed in (FIG. 21).

Patients who have duodenogastric reflux may have a gastric juice in this dangerous intermediate pH range and will therefore generate a carcinogenic milieu for esophageal adenocarcinoma. For that person to actually develop adenocarcinoma requires that reflux results in effective doses of the carcinogen delivered to the target cell, i.e. intestinal metaplasia (FIG. 20). If this does not happen, adenocarcinoma does not occur, whatever the degree of carcinogenesis in the gastric juice. If there has been a natural increase in the tendency to alkalinize gastric juice by increasing duodenogastric reflux, this can explain the historic increase in the incidence of adenocarcinoma. However, whatever carcinogenic tendency exists in the patient, acid suppressive drug therapy will cause a tilt in the equilibrium that promotes and increases the risk of carcinogenesis, as described in (FIG. 21).

First, the increased alkalinization will tend to induce intestinal metaplasia of cardiac mucosa at increasingly distal regions of the esophagus, as discussed in relation to (FIG. 19), bringing the target cell closer to the gastroesophageal junction and carcinogen. Secondly, the alkalinization of gastric juice will increase the generation of carcinogens from bile acids (FIG. 21), increasing the dose of carcinogen in gastric juice and driving the point of effective carcinogenesis in the esophagus more proximally. The combination of the two factors brings the target cell and carcinogen into increasingly greater contact in the patient using acid suppressive drugs.

Diagnosis Based on the Recognition of Different Risk Levels in the Epithelia of Columnar Lined Esophagus

As previously discussed, the current invention is based on based on the understanding that adenocarcinoma is caused when components of the gastric refluxate, most likely acid with possible contribution by proteolytic enzymes and bile salts, damage the squamous epithelium of the esophagus. This causes changes in the squamous epithelium such as basal cell hyperplasia and eosinophil infiltration. It also results in separation of the squamous epithelial cells (dilated intercellular spaces) which permits luminal molecules of size up to 20 kD to penetrate the epithelium. (Tobey N A, et al., 1996; Tobey N A, et al., 2004; and Villanacci V, Grigolato P G, Cestari R, et al. Dilated intercellular spaces as markers of reflux disease: Histology, semiquantitative score and morphometry upon light microscopy. Digestion 2001; 64:1-8, the disclosure of which is incorporated herein by reference.) In turn, luminal molecules reaching the proliferating stem cells in the basal layer of the epithelium are likely responsible for producing a genetic switch from squamous to columnar differentiation, resulting in a metaplastic columnar epithelium (FIG. 6).

However, columnar metaplasia of the esophageal squamous epithelium is merely the beginning of the reflux-adenocarcinoma sequence (FIG. 6). There is nothing in the gastric refluxate that is carcinogenic to esophageal squamous epithelium; reflux is not associated with squamous carcinoma. As soon as columnar metaplasia occurs, undetermined molecules in the refluxate begin to exert a carcinogenic influence on the metaplastic epithelium. This occurs as a phenomenon that may be independent from molecules causing injury to the metaplastic columnar epithelium.

Luminal molecules that are injurious to the columnar epithelium direct the evolution of the metaplastic columnar epithelium. Initially, the metaplastic epithelium consists of undifferentiated mucous cells only (cardiac mucosa). In a low damage environment, cardiac mucosa evolves into oxynto-cardiac mucosa. This is characterized by the appearance of parietal cells, a phenotypic expression that must correlate with the expression of yet undetermined genetic signals that direct a gastric-type of differentiation. Oxynto-cardiac mucosa is more resistant to gastric juice, showing less inflammation than cardiac mucosa (FIG. 3), and almost never undergoes intestinal metaplasia, dysplasia or adenocarcinoma. It therefore represents a benign component of the columnar lined esophagus.

In a high damage environment, cardiac mucosa does not generate parietal cells, remaining as cardiac mucosa (mucous cells only—FIG. 22) or developing goblet cells to become intestinal metaplastic epithelium (FIG. 4). Intestinal metaplasia represents the phenotypic expression of aberrant genetic signals such as Cdx1 and Cdx2. (Phillips R W, Frierson H F, Moskaluk C A. Cdx2 as a marker of epithelial intestinal differentiation in the esophagus. Am J Surg Pathol 2003; 27:1442-1447; and Silberg D G, Swain G P, Suh E R, Traber P G. Cdx1 and Cdx2 expression during intestinal development. Gastroenterol 2000; 119:961-971, the disclosures of which are incorporated herein by reference.) These normally drive differentiation in the intestine and colon and are not expressed in the normal esophagus or stomach. (Silberg, et al., 2000, previously cited.) The intestinal type of metaplastic columnar epithelium of the esophagus is the only epithelium that is susceptible to the action of undetermined carcinogenic molecules in the gastric refluxate. In a carcinogenic environment, the intestinal epithelium undergoes progression in the reflux-adenocarcinoma sequence, developing low grade dysplasia, high grade dysplasia and finally adenocarcinoma (FIG. 6).

The columnar lined esophagus is therefore an epithelium that is heterogeneous in different patients. It has varying amount of intestinal epithelium that is at high risk for adenocarcinoma, cardiac mucosa that is at risk for undergoing intestinal metaplasia, and oxynto-cardiac mucosa which is not at risk. Recognizing the variation of cancer risk within the epithelial types of columnar lined esophagus and combining this with the correlation between length of columnar lined esophagus and severity of reflux permits the development of a practical method of classification of the population in terms of reflux induced damage, as discussed in Table 7, below. (Chandrasoma P. Pathological basis of gastroesophageal reflux disease. World J Surg 2003; 27:986-993, the disclosure of which is incorporated herein by reference.)

TABLE 7
CLASSIFICATIONS OF RISK OF ADENOCARCINOMA

| Risk Group | Definition | SubGroups |
|---|---|---|
| Grade Zero: No Risk | No cardiac or intestinal | Group 0a: NORMAL: Only squamous |
| Of Adenocarcinoma | metaplasia in any | epithelium and gastric oxyntic mucosa. |
| (55-65%) | biopsy. | Group 0b: COMPENSATED REFLUX: |
| | | Oxynto-cardiac mucosa as the only |
| | | epithelium in the metaplastic columnar |
| | | esophagus. |
| Grade 1: Reflux | Cardiac mucosa (reflux | Group 1a: MILD REFLUX DISEASE: |
| Disease (30-45%) | carditis) present; | Endoscopy normal. |
| | oxynto-cardiac mucosa | Group 1b: MODERATE REFLUX DISEASE: |
| | also present in most | Endoscopy shows a columnar lined |
| | cases. | esophagus <2 cm long. |
| | | Group 1c: SEVERE REFLUX DISEASE: |
| | | Endoscopy shows a columnar lined |
| | | esophagus >2 cm long. |
| Grade 2: Barrett | Intestinal metaplasia | Group 2a: MICROSCOPIC BARRETT |
| Esophagus (5-15%) | (goblet cells) present | ESOPHAGUS: Endoscopy normal. |
| | in cardiac mucosa; | Group 2b: SHORT SEGMENT BARRETT |
| | non-intestinalized | ESOPHAGUS: Endoscopy shows a |
| | cardiac mucosa and | columnar lined esophagus <2 cm long. |
| | oxynto-cardiac mucosa | Group 2c: LONG SEGMENT BARRETT |
| | also present in most | ESOPHAGUS: Endoscopy shows a |
| | cases. | columnar lined esophagus >2 cm long. |
| Group 3: Neoplastic | Dysplasia or | Group 3a: Low grade dysplasia. |
| Barrett Esophagus | adenocarcinoma | Group 3b: High grade dysplasia. |
| | present. | Group 3c: Adenocarcinoma. |

Within each group is recognized a subgroup that is based on the length of columnar lined esophagus present which is an accurate predictor of severity of reflux-induced damage of the esophagus. The percentage of the population that is probably within each group is given in parentheses.

The most important feature of this classification is that, for the first time, it provides a method to identify the approximately 55-65% of the population that has no risk of adenocarcinoma.

Diagnosis of Intestinal Metaplasia

In all patients with metaplastic columnar epithelium who have intestinal metaplasia (i.e. Barrett esophagus), there is a highly predictable pattern of zonation of the three epithelial types. (Paull A, et al., 1976; and Chandrasoma P T, Der R, Dalton P, et al. Distribution and significance of epithelial types in columnar lined esophagus. Am J Surg Pathol 2001; 25:1188-1193, the disclosure of which is incorporated herein by reference.) The intestinal metaplasia occurs at the proximal end immediately distal to the squamo-columnar junction and oxynto-cardiac mucosa tends to occur in the most distal segment. This zonation exists irrespective of the length of the metaplastic columnar lined segment. Once it arises at the squamo-columnar junction, intestinal metaplasia progressively extends distally to involve increasing amounts of the metaplastic segment. In almost every case, however, there is residual non-intestinalized cardiac mucosa and oxyntocardiac mucosa distal to the intestinal epithelium.

Accordingly, in one embodiment of the invention a diagnostic method for intestinal metaplasia in a segment of columnar lined esophagus would involve a biopsy at the area of columnar epithelium immediately distal to the squamo-columnar junction. (Chandrasoma P T, et al., 2001, previously cited.) This method will have the highest yield of intestinal metaplasia. The situation is different for surveillance biopsies in Barrett esophagus that are designed to detect dysplasia. Dysplasia occurs randomly throughout the segment of metaplastic columnar epithelium and surveillance biopsies must follow a standard protocol that covers the full extent of the columnar lined esophagus.

Diagnosis of Barrett Esophagus

The definition of Barrett esophagus has evolved over the years and will continue to do so until the definition in accordance with the current invention is adopted. In the pre-1976 era, Barrett esophagus was an endoscopic diagnosis made when there was columnar lining in the distal esophagus that exceeded the 3 cm that was considered normal. When Paull et al described three epithelial types in columnar lined esophagus in 1976, this definition did not change, but Barrett esophagus was classified histologically into three types: junctional (or cardiac), fundic (or oxynto-cardiac) and specialized (or intestinal). (Paull A, et al., 1976, previously cited.) In the early 1980s, evidence emerged that only the intestinal histologic type of Barrett esophagus predisposed to the development of adenocarcinoma. (Haggitt R C, Tryzelaar J, Ellis F H, et al. Adenocarcinoma complicating columnar epithelium-lined (Barrett's) esophagus. Am J Clin Pathol 1978; 70:1-5; Haggitt R C, Dean P J. Adenocarcinoma in Barrett's epithelium. In Spechler S J, Goyal R K (eds): Barrett's Esophagus: Pathophysiology, Diagnosis, and Management. New York, Elsevier Science Publishing, 1985, pp 153-166; and Reid B J, Weinstein W M. Barrett's esophagus and adenocarcinoma. Ann Rev Med 1987; 38:477-492, the disclosures of which are incorporated herein by reference.) After this, the diagnosis of Barrett esophagus required not only the presence of >3 cm of columnar lined esophagus, but also the histologic demonstration of intestinal metaplasia. In the early 1990s, Spechler showed that segments of columnar lined esophagus shorter than 3 cm frequently showed intestinal metaplasia. (Spechler S J, Zeroogian J M, Antonioli D A, Wang H H, Goyal R K. Prevalence of metaplasia at the gastro-esophageal junction. Lancet 1994; 344:1533-1536, the disclosure of which is incorporated herein by reference.) When this was suspected to be pre-malignant, the concept of short segment Barrett esophagus was developed. (Rudolph R E, Vaughan T L, Storer B E et al. Effect of segment length on risk for neoplastic progression in patients with Barrett esophagus. Ann Intern Med 2000; 132:612-620, the disclosure of which is incorporated herein by reference.)

This then represents the present state of the definition of Barrett esophagus. According to the American Gastroenterological Association, Barrett esophagus requires the presence of intestinal metaplasia in a biopsy taken from an endoscopically visualized abnormal segment of columnar lined esophagus. (Sampliner R E, Practice Parameters Committee of the American College of Gastroenterology. Practice guidelines on the diagnosis, surveillance, and therapy of Barrett's esophagus. Am J Gastroenterol 1998; 93:1028-1032, the disclosure of which is incorporated herein by reference.)

Herein Barrett esophagus is defined as the occurrence of intestinal metaplasia in metaplastic columnar epithelium of the esophagus, i.e., Barrett esophagus is esophageal intestinal metaplasia. With the histologic definitions previously discussed, the diagnosis for Barrett esophagus in accordance with the current invention is simple: it is the occurrence of goblet cells in cardiac mucosa (FIG. 4). As previously discussed, oxynto-cardiac mucosa never undergoes intestinal metaplasia. Intestinal metaplasia occurring in gastric oxyntic mucosa is gastric intestinal metaplasia complicating atrophic gastritis; it has nothing to do with the esophagus or reflux. In short, it is only if, in contrast with the current invention, cardiac mucosa is treated as a normal epithelium of the stomach that it becomes impossible to diagnose Barrett esophagus.

In accordance with the current invention, the diagnosis of Barrett type intestinal metaplasia is made when one or more definite goblet cells are present in cardiac mucosa in an H&E stained section. A goblet cell has a round vacuole that distends the lateral borders of the cell and commonly has a basophilic tinge. Goblet cells must be distinguished from “pseudo-goblet cells” which are mucin distended cardiac mucosal cells. (Chandrasoma P T, et al., 2001, previously cited.) In this technique it is preferred not to use Alcian blue staining because Alcian blue produces positive staining in mucin distended pseudo-goblet cells (“columnar blue cells”) in addition to goblet cells, and there is a considerable risk of over diagnosis of Barrett esophagus.

In most cases, the diagnosis of Barrett esophagus in accordance with the current invention is easy and clear cut, as summarized in Table 8, below. However, difficulty may arise in patients who have both reflux disease of the esophagus and gastritis. In most of these cases, the gastritis that affects oxyntic mucosa is typically an active chronic superficial gastritis with a relatively uninvolved glandular zone containing parietal cells. This is easy to distinguish from Barrett type intestinal epithelium where there are no parietal cells, the glands are disorganized and the inflammation occurs throughout the mucosa. Considerably greater difficulty arises when a short segment of Barrett type intestinal metaplasia of the esophagus coexists with multifocal atrophic gastritis with gastric intestinal metaplasia. Atrophic gastric mucosa loses its parietal cells and becomes a mucous cell only mucosa in the end stage of the disease and can superficially resemble cardiac mucosa. However, cardiac mucosa with Barrett type intestinal metaplasia is a reactive and often villiform epithelium compared to the flat epithelium of atrophic gastritis, permitting accurate differentiation in most cases. Some authorities suggest the use of special mucin stains such as high-iron diamine and immunoperoxidase staining with antibodies such as DAS-1 (7E₁₂H₁₂ monoclonal antibody) and cytokeratin 7 and 20. (Das K M, Prasad I, Garla S, Amenta P S. Detection of a shared colon epithelial epitope on Barrett epithelium by a novel monoclonal antibody. Ann Intern Med 1994; 120:753-756; and Ormsby A H, Goldblum J R, Rice T W, et al. Cytokeratin subsets can reliably distinguish Barrett's esophagus from intestinal metaplasia of the stomach. Hum Pathol 1999; 30:288-294, the disclosure of which is incorporated herein by reference.) As previously discussed stains are often unreliable, accordingly the current invention is still directed to morphological techniques as the best means of differentiating between the two entities.

TABLE 8
DIAGNOSIS OF REFLUX DISEASE AND GASTRITIS

| Case | Pathological Findings |
|---|---|
| Reflux Disease of the | Reflux changes in squamous epithelium +/−; |
| Esophagus without Gastric | Metaplastic columnar epithelium - oxynto-cardiac, cardiac |
| Abnormality | and/or Barrett type intestinal metaplasia; |
| | Normal gastric oxyntic and pyloric mucosa. |
| Gastritis without Reflux | Normal squamous epithelium; |
| Disease of the Esophagus | Absence of metaplastic columnar epithelium; no oxynto- |
| | cardiac, cardiac or Barrett type intestinal metaplasia; |
| | Gastritis involving oxyntic and/or pyloric epithelium, with or |
| | without atrophy and gastric intestinal metaplasia. |
| Simultaneous Reflux | Reflux changes in squamous epithelium +/−; |
| Disease of the Esophagus | Metaplastic columnar Epithelium - oxynto-cardiac, cardiac and |
| and Gastritis | Barrett type intestinal metaplasia; |
| | Gastritis involving oxyntic and/or pyloric epithelium, with or |
| | without atrophy and gastric intestinal metaplasia. |

As previously discussed, many experts in the field still believe that intestinal metaplasia arises directly from squamous epithelium, and that the only columnar lined esophagus that is abnormal is intestinal metaplasia. As a result, there is a general tendency to treat a biopsy from columnar lined esophagus in a manner similar to a biopsy from a mass lesion. If a biopsy from a mass lesion shows cancer, it can be assumed that the entire mass lesion is composed of that cancer. Gastroenterologists view a segment of columnar lined esophagus and take multiple biopsies; if any one biopsy shows any amount of intestinal metaplasia, the assumption is made that the entire segment is lined by intestinal metaplasia. This becomes Barrett esophagus of a length that is determined by the endoscopically determined length of columnar lined esophagus.

However, this technique fails to recognize that the amount of intestinal metaplasia varies greatly in different columnar lined segments of equal or different lengths, as shown in (FIG. 22). It is possible that a long segment has very few goblet cells while a short segment has more. To try to equate risk of cancer by the length of the entire epithelium than by the number of target cells, as suggested by the current invention is an error. This error results from the fact that most clinicians never look at histologic slides, pathologists fail to communicate, and cardiac and oxyntocardiac mucosa are simply ignored as being irrelevant. This misinformation creates havoc in treatment and assessment of patients with Barrett esophagus.

(a) Ablation of Barrett Esophagus

One detrimental aspect of the current diagnosis methodology is that it results in over-treatment when Barrett esophagus is ablated. Specifically, when a decision is taken to ablate Barrett esophagus, the objective is to ablate the entire length of columnar lined esophagus, not just the at-risk intestinal metaplastic epithelium. To add risk and morbidity by ablating oxyntocardiac mucosa is not helpful because the oxyntocardiac mucosa that is to be replaced with squamous epithelium is just as harmless.

Accordingly, in one embodiment the invention is drawn to a diagnosis and treatment methodology which includes mapping the epithelia so that only the at-risk intestinal epithelium, which may actually be quite limited within the columnar lined segment, is ablated, thereby greatly decrease the amount of mucosa requiring ablation.

(b) Assessment of Regression of Barrett Esophagus

Many studies have been conducted that look for evidence of “regression of Barrett epithelium”. The way these studies are designed is to define the study group by the presence of columnar lined esophagus which has intestinal metaplasia, measure the endoscopic length of the columnar epithelium, and then evaluate whether the columnar epithelium has decreased in amount due to the growth of squamous epithelium in areas previously covered by columnar epithelium. It has been discovered that this is not regression in any sense of the word. Instead, it has been determined that the surface growth of squamous epithelium is often associated with columnar epithelium in the lamina propria under the squamous surface, as described in (FIG. 23). This columnar epithelium has been shown to undergo dysplasia and malignancy while still covered by squamous epithelium, as shown in (FIG. 24). Therefore, a squamous surface is not evidence of regression.

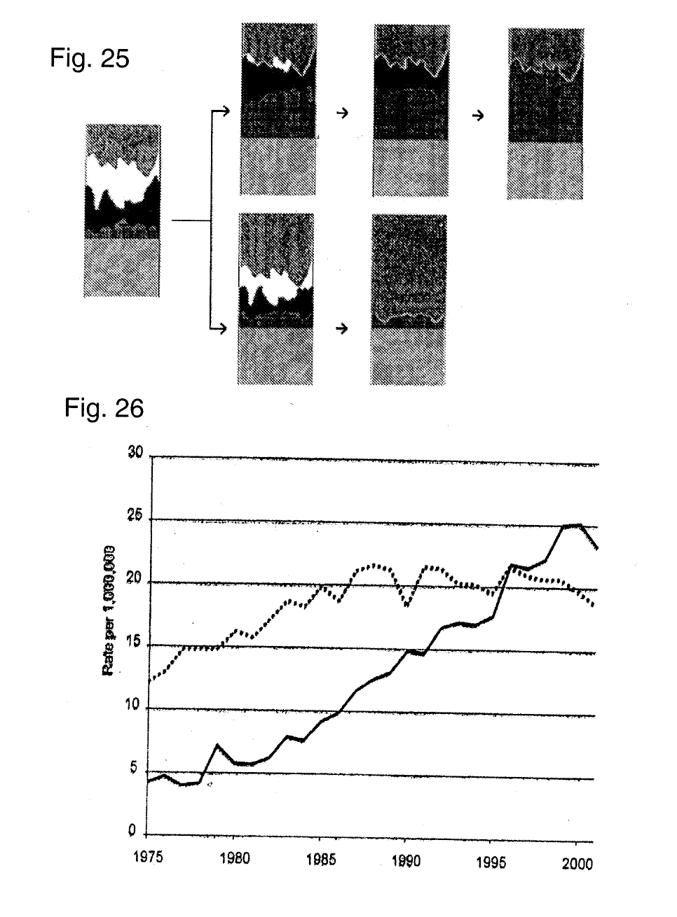

Instead, the critical measure of cure or regression in Barrett esophagus is a reversal or decrease in the number of intestinal metaplastic cells, because this is the only target cell for carcinogenesis (FIG. 25). A columnar epithelium of equal length that has less intestinal metaplasia and more oxyntocardiac mucosa is an epithelium that has fewer target cells for carcinogenesis and therefore less dangerous. Accordingly, the aim of the current treatment regime is to reverse intestinal metaplasia to cardiac mucosa and to convert cardiac mucosa to oxyntocardiac mucosa. This would be a true regression of Barrett esophagus.

Any such treatment should be combined with diagnostic studies that evaluate changes in different epithelial types resulting from various treatment modalities. These evaluative techniques should be focused on examining the changes in density of goblet cells (any decrease is good) and parietal cells (any increase is good) within the segment of columnar lined esophagus, not at squamous replacement on the surface, as used in conventional techniques.

Treatment Methodologies

As previously discussed, pharmaceutical research in reflux disease has been single-mindedly directed toward the development of ever-improving acid suppressive drugs to treat symptoms and cause healing of erosive reflux disease. This has been remarkably successful in achieving its goal. Acid suppression with drugs is now theoretically capable of producing almost complete suppression of acid secretion. Acid suppression has essentially reversed squamous epithelial damage, resulting in control of symptoms, improving quality of life and preventing complications such as complex strictures and severe uncontrolled ulceration.

Because of the “success” of acid suppressive drugs in treating reflux disease there has been almost no effort by the pharmaceutical drug industry to develop alternate agents to impact reflux disease. These drugs are so profitable because of their widespread use that the thrust of competition has been merely to produce ever increasingly effective acid suppression. In short, the medical community has placed all its pharmaceutical eggs for treating reflux disease in one basket.

Even as acid suppression has effectively controlled the squamous epithelial damage and improved the life of many patients, there has been an alarming increase in the incidence of reflux-induced adenocarcinoma. In 1992, Rodger Haggitt suggested this was an epidemic (Haggitt R C. Adenocarcinoma in Barrett's esophagus: a new epidemic? Hum Pathol 1992:23:475-476, the disclosure of which is incorporated herein by reference); Devesa et al (1998) reported a 350% increase in incidence in white males in the United States over the two decades preceding 1995. (Devesa S S, Blot W J, Fraumeni J F. Changing patterns in the incidence of esophageal and gastric carcinoma in the United States. Cancer 1998; 83:2049-2053, the disclosure of which is incorporated herein by reference.) A more recent study by Pohl and Welch (2005) showed that the overall esophageal adenocarcinoma incidence has continued to rise. (Pohl H, Welch H G. The role of over diagnosis and reclassification in the marked increase of esophageal adenocarcinoma incidence. J Natl Cancer Inst. 2005; 97:142-6, the disclosure of which is incorporated herein by reference.) In this report, the incidence increased nearly six-fold between 1975 and 2001 from 4 to 23 cases per million (FIG. 26).

Despite this alarming statistical trend, there has been a vehement denial that there is any possible connection between the increasing usage of acid suppressive agents and the increasing incidence of adenocarcinoma. Herein it is proposed that the epidemiologic data suggest (Lagergren J, Bergstrom R, Lindgren A, Nyren O. Symptomatic gastroesophageal reflux as a risk factor for esophageal adenocarcinoma. N Engl J Med 1999; 340:825-831, the disclosure of which is incorporated herein by reference) that the use of these drugs actually increases the risk of cancer, and that accordingly the emphasis on these drugs has been misplaced. It is proposed that the reason for the failure of conventional treatments to address the growing “epidemic” of adenocarcinoma is that these conventional treatments are based on a naïve belief that acid damage of squamous epithelium causes intestinal metaplasia and then carcinoma.

In the discussion of the three critical events that are necessary for carcinogenesis above, it is proposed that two of these events are promoted by increased alkalinity of gastric juice. In short, that the occurrence and extent of intestinal metaplasia in esophageal cardiac mucosa increase as alkalinitiy of gastric juice increases (FIG. 19). Moreover, it is also proposed that the effective carcinogenic concentration in gastric juice, and the height in the esophagus to which the carcinogenicity reaches is also increased by increasing gastric alkalinization (FIGS. 20 and 21). Acid suppressive agents function by suppressing acid secretion in the stomach and always cause an increased alkalinity in gastric juice if they have any efficacy. As such, it is further suggested that these acid suppressive agents actually promote carcinogenesis in Barrett esophagus.

It should be understood that the use of acid suppressive drugs is unlikely to be the entire reason for the increasing incidence of esophageal adenocarcinoma over the past five decades. In fact, most patients who present with esophageal adenocarcinoma have never taken acid suppressive medications. This suggests that there has been an increase in the yet unknown natural factors that are responsible for Barrett esophagus and esophageal adenocarcinoma. However, while acid suppressive drugs are not likely to be the main culprits, it is proposed that they are promoters, enhancing carcinogenesis and increasing the risk of cancer in patients taking these drugs for the treatment of reflux disease.

Moreover, the cancer-promoting effect of acid suppressive drugs need only be slight to have a significant numerical impact in the incidence of reflux-induced adenocarcinoma. Adenocarcinoma occurs only in a very small percentage of patients with reflux disease. However, the population at risk is large; 35-45% of the population has cardiac mucosa, which is the definition of reflux disease used herein. 35-45% of the population of the USA represents over 100,000 million people. Even a slight tilt in the balance to increase the conversion of cardiac mucosa to intestinal metaplasia would have the effect of greatly increasing the prevalence of Barrett esophagus. Acid suppression, by causing a slight shift of the pH in gastric juice can have this effect. It can also explain the increased prevalence of Barrett esophagus over the past five decades.

In summary it is proposed that a slight tilt in the balance to increase conversion of bile acid derivatives to carcinogenic agents (FIG. 21) will have the effect of significantly increasing the absolute number of Barrett esophagus patients who progress to cancer. The six-fold increase in adenocarcinoma that has occurred in the USA over the past three decades requires only a minimal shift in the gastric pH resulting from acid suppression, assuming all other factors remain equal. The main factors causing Barrett esophagus and adenocarcinoma are the occurrence of adequate gastroesophageal reflux and a carcinogenic environment in the stomach. Acid suppressive drugs, by causing a very slight tilt in the gastric juice pH towards alkaline, enhances this natural carcinogenic effect. Patients with adequate carcinogenicity increase their risk and rate of progression; patients in the borderline range of carcinogenicity who would never have developed cancer receive an adequate nudge to tilt the balance. This explains how most reflux-induced cancers occur in asymptomatic people who have never taken acid suppressive agents develop cancer and how acid suppressive drugs increase the risk of cancer.

The only current alternative to controlling symptoms of reflux disease, which can be debilitating to sufferers, is anti-reflux surgery. However, there are not sufficient trained surgeons at the present time to perform the required numbers of these operations

Novel Pharmaceutical Targets for Preventing Adenocarcinoma

Rather than treating the systems of reflux disease, in one embodiment of the current invention a treatment regime is proposed that would prevent the molecular interactions that are responsible for the cellular transformations that take squamous epithelium through cardiac mucosa into intestinal metaplasia (FIG. 14, Table 6).

The technology for cell culture has reached an advanced stage. Fitzgerald et. al. showed the feasibility of growing human cells in culture and seeing the effects of exposure to acid and bile salt derivates. (Fitzgerald R C, Omary M B, Triadafilopoulos G. Dynamic effects of acid on Barrett's esophagus: an ex-vivo proliferation and differentiation model. J Clin Invest 1996; 98:2120-2128, the disclosure of which is incorporated herein by reference.) They measured the response of cells grown from human Barrett epithelium in terms of proliferative rate and differentiation by evaluating expression of proliferative cell nuclear antigens and villin. More recently, Kazumori et al (2005), tested rat keratinocytes in culture against different bile salt derivatives, showing that cholic acid and dehydrocholic acid increased Cdx2 promoter activity in keratinocytes and induced production of intestinal type mucin, suggesting that these bile salt derivatives may be involved in columnar metaplasia. (Kazumori H, Ishihara S, Rumi M A, Kadowaki Y, Kinoshita Y. Bile acids directly augment caudal-related homeobox gene Cdx2 expression in esophageal keratinocytes in Barrett's epithelium. Abstract ID: S1674, DDW, 2005, the disclosure of which is incorporated herein by reference.)

These new technologies have permitted researchers to demonstrate that Cdx2 promotion was induced via NF-kB binding sites on the surface of the cell. However, the focus of these studies is again on squamous epithelium and intestinal metaplasia. Currently no research is being directed at testing cardiac mucosa.

In summary, the ultimate goal of treating and preventing adenocarcinoma relies on the recognition of the pathogenesis of each cellular event in terms of a molecular event. Determining these molecular events will permit a specific intervention at each critical step in the carcinogenic sequence that in turn will prevent the progression to adenocarcinoma. Accordingly, the following events form the targets for pharmaceutical interventions:

(a) Squamous Epithelial Transformation to Cardiac Mucosa

The first change that takes the esophageal epithelial cells towards susceptibility to carcinogenesis (i.e. intestinal metaplasia) is the conversion of squamous epithelium to cardiac mucosa. This is an almost invariable change when there is reflux as it occurs in virtually all people with reflux as a very early cellular event. Accordingly, the presence of metaplastic columnar epithelium is by far the most sensitive indicator of reflux disease.

2. Cardiac mucosa is commonly seen in children with reflux (Hassall E. Columnar lined esophagus in children. Gastroenterol Clin N Amer 1997; 26:533-548; and Glickman J N, Fox V, Antonioli D A, Wang H H, Odze R D. Morphology of the cardia and significance of carditis in pediatric patients. Am J Surg Pathol 2002; 26:1032-1039, the disclosures of which are incorporated herein by reference) and occurs above the anastomotic line in patients who have had an esophagectomy within a short period after surgery (Dresner S M, Griffin S M, Wayman J, Bennett M K, Hayes N, Raimes S A. Human model of duodenogastro-oesophageal reflux in the development of Barrett's metaplasia. Br J Surg 2003; 90:1120-1128; and Lord R U N, Wickramasinghe K, Johansson J J, DeMeester S R, Brabender J, DeMeester T R. Cardiac mucosa in the remnant esophagus after esophagectomy is an acquired epithelium with Barrett's like features. Surgery 2004; 136:633-640, the disclosures of which are incorporated herein by reference). Cameron et al produced data that suggested that the length of columnar lined esophagus increases to its maximum at a relatively early age and then remains static.

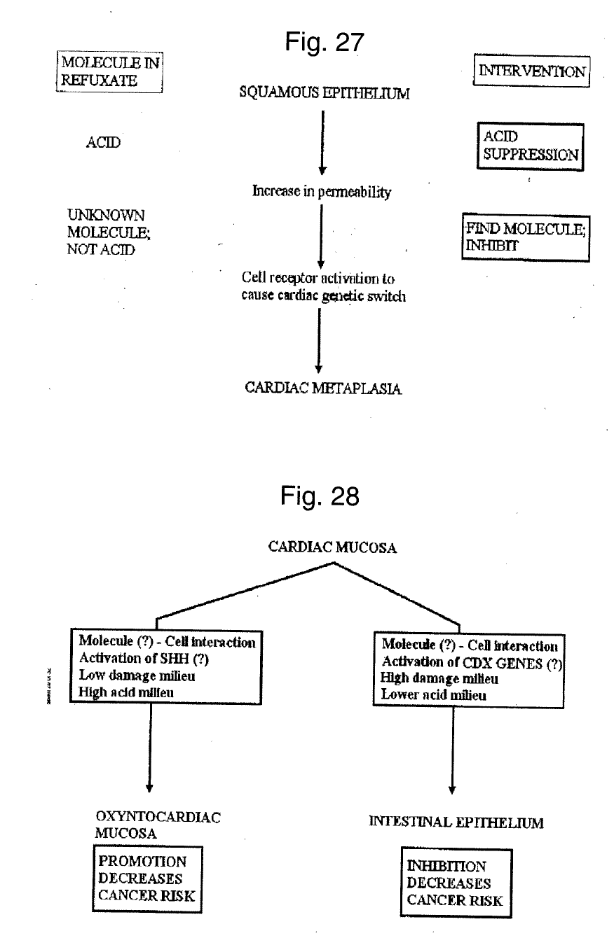

This data suggests that the transformation of squamous epithelium to cardiac mucosa is a relatively rapid and essentially invariable change in patients with significant reflux. Acid is necessary for the process because it is the agent that damages the squamous cells, increasing the spaces between them and permitting refluxate molecules to enter the epithelium and access the pool of proliferating progenitor cells in the basal region (FIG. 27). Because cardiac mucosal metaplasia occurs very early, acid suppression can only be effective if it is used in everyone in the population from an early stage in life. This is obviously not feasible.

As discussed above, the cause of cardiac metaplasia is posited to be caused by an interaction between a larger refluxate molecule and a cell receptor. By identifying and preventing the cell interaction that activates the genetic switch that converts squamous epithelium to cardiac mucosa it would be possible to design drugs to stop the cellular transformation that leads to reflux disease. One way to make the necessary determination would be to expose the esophageal squamous epithelial cells in tissue culture to various molecular components in gastric juice and identify the occurrence of columnar (mucous) cell transformation. The fact that squamous to cardiac metaplasia is inevitable in patients with reflux means that the molecule causing this change is ubiquitous in gastric juice. Examining differences between the activated genes in the original squamous and transformed columnar epithelial cell would then lead to the detection of the actual gene activated in the metaplastic switch. However, despite its potential, it is unlikely that elucidation of this molecular reaction and genetic switch could be used in the vast majority of patients because cardiac mucosal metaplasia occurs early and with great rapidity.

Once cardiac metaplasia occurs in a patient with reflux disease, it is unlikely that reversal to squamous epithelium will occur naturally. Accordingly, the current invention is also directed to pharmaceutical interventions that change the milieu and can reverse this reaction.

In this embodiment of a treatment regime for reflux disease the target would be the squamous cells. Specifically, squamous islands appear in columnar lined esophagus after both acid suppressive therapy and anti-reflux surgery, suggesting that reversal of metaplasia is feasible. Elucidating the molecular mechanism of cardiac metaplasia may provide more effective methods of reversal to squamous epithelium. One problem with such reversal is that surface squamous transformation may result in the entrapment of metaplastic glandular elements in the lamina propria. These are hidden under the squamous surface at endoscopy and gross examination but may retain the potential to progress to intestinal metaplasia, dysplasia and adenocarcinoma (FIGS. 23 and 24).

One interesting and counter-intuitive mechanism for treating symptomatic reflux without the use of acid suppressive agents is to promote the conversion of reflux-damaged squamous epithelium to cardiac mucosa by a drug that stimulates this conversion. In the context of the current invention esophageal pain is postulated to be the result of stimulation of nerve endings in squamous epithelium; columnar epithelia are believed to be much less sensitive. As a result, conversion of pain-generating acid-damaged squamous epithelium to cardiac mucosa can have the effect of decreasing heartburn. Treatments to prevent intestinal metaplasia in cardiac mucosa and promote conversion of cardiac mucosa to oxyntocardiac mucosa can also be a highly effective and practical pain-relieving mechanism in reflux disease. In short, from the point of view of pain, columnar epithelia are better than squamous epithelium.

(b) Cardiac Mucosal Transformation to Oxyntocardiac Mucosa

The conversion of cardiac mucosa to oxyntocardiac mucosa represents another mechanism of “cure” of reflux disease because oxyntocardiac mucosa is a stable epithelium that does not progress to intestinal metaplasia. In this embodiment of the invention, the patient is removed from the carcinoma sequence shown in (FIG. 14). As previously discussed there is evidence that oxyntocardiac mucosal transformation of cardiac mucosa is the result of the following conditions being present: (a) a low damage environment; this exists in patients with mild reflux, in the distal part of the esophagus when there is continuous rather than pulse (or intermittent) exposure of the epithelium to the refluxate (Fitzgerald et al., 1996, previously cited), and after successful anti-reflux surgery when reflux has ceased; and (b) when the pH is low; parietal cell generation in cardiac mucosa appears to be promoted by an acid environment. There is also evidence that the genetic system whose activation leads to conversion of cardiac to oxyntocardiac mucosa is the Sonic Hedgehog gene system (FIG. 30).

If the cardiac to oxyntocardiac conversion is pH based and promoted by low pH, acid suppression will be expected to have an inhibitory effect on this change. The current invention posits that acid suppression has actually caused an increase in cancer. A number of treatments arise from this situation: (a) using alternate drugs that can relieve symptoms and complications of reflux like erosions, ulcers and strictures but that do not work by suppressing acid, i.e., controlling the disease without increasing the gastric pH; (b) administering drugs that simulate the action of acid on cardiac mucosa that promotes its conversion to oxyntocardiac mucosa without causing squamous epithelial damage would also serve as an agonist to this change; (c) because the natural alkalinizing agent of gastric juice is duodenogastric reflux, administering drugs that are designed to prevent duodenogastric reflux or antagonize the alkalinity of duodenogastric refluxate need to be developed and tested; and (d) administering acid suppressive drugs with appropriate caution in the management of symptomatic reflux disease because these can theoretically prevent the cardiac to oxyntocardiac transformation.

Specific agents that target the molecular mechanism and molecular agent that causes the transformation of cardiac to oxyntocardiac mucosa have great potential value. The development of such a drug will involve growing esophageal cardiac epithelial cells in tissue culture and exposing these to a variety of conditions and different molecules normally present in the gastric juice. The end-point of the experiment is the detection of parietal cell differentiating or Sonic Hedgehog gene activation. Once the exact molecular agent is identified, drugs can be developed that can replicate the action of this molecule and promote this transformation. This will have the effect of converting cardiac mucosa to oxyntocardiac mucosa and preventing adenocarcinoma.

TABLE 9
THERAPEUTICS FOR REFLUX-INDUCED ADENOCARCINOMA

| Point of Intervention | Possible agents |
|---|---|
| Inhibit cardiac mucosal | (a) Acid suppression; |
| metaplasia of squamous | (b) Find new drugs that increase |
| epithelium | squamous epithelial resistance to acid |
| | damage; and |
| | (c) Find basis of genetic switch and |
| | develop an antagonistic drug. |
| Reverse cardiac metaplasia and | Find basis of genetic switch and |
| induce squamous differentiation | develop drug to reverse it. |
| Promote cardiac metaplasia in | Find basis of genetic switch and |
| acid-damaged squamous | develop drug to promote it; can |
| epithelium | relieve heartburn. |
| Promote the conversion of | (a) Find alternate new drugs to relieve |
| cardiac mucosa to oxyntocardiac | reflux symptoms without acid |
| mucosa | suppression; |
| | (b) Find drugs that simulate acid |
| | without causing squamous epithelial |
| | damage; |
| | (c) Decrease the alkalinizing effect of |
| | duodenogastric reflux; and |
| | (d) Find basis of genetic switch and |
| | develop a drug that activates or |
| | simulates the causative agent. |
| Inhibit the conversion of cardiac | (a) Find alternate new drugs to relieve |
| mucosa to intestinal metaplasia | reflux symptoms without acid |
| | suppression; |
| | (b) Decrease the alkalinizing effect of |
| | duodenogastric reflux; and |
| | (c) Find basis of genetic switch and |
| | develop an antagonistic or inhibitory |
| | drug. |
| Reverse intestinal metaplasia to | Find basis of genetic switch and |
| cardiac mucosa | develop a drug to reverse the change. |

(c) Cardiac Mucosal Transformation to Intestinal Epithelium

The conversion of cardiac mucosa to intestinal epithelium represents progression of reflux disease in the adenocarcinoma sequence. Unlike cardiac mucosa, intestinal metaplastic epithelium is the target epithelium for carcinogens (FIG. 14). The current invention provides evidence that the amount of intestinal epithelium in columnar lined esophagus has increased in the past three decades and suggests that this may be a reason for the increase in esophageal adenocarcinoma.

There is evidence that intestinal transformation of cardiac mucosa is the result of the following conditions being present (FIG. 30):

(a) A high damage environment; this exists in patients with severe reflux, in the more proximal part of the esophagus when there is pulse (or intermittent) rather than continuous exposure of the epithelium to the refluxate (Fitzgerald et al., 1996, previously cited).

(b) When the pH is high; goblet cell generation in cardiac mucosa appears to be promoted by a less acid environment. This is seen in patients who have acid suppressive drug treatment, providing the basis for the observed association between acid suppressive drug use and the increased incidence of adenocarcinoma. There is also evidence that the genetic system whose activation leads to conversion of cardiac to intestinal epithelium is the CdX gene system (FIG. 14).

Provided that the cardiac to intestinal conversion is pH based and promoted by a high pH, acid suppression will be expected to have a stimulating effect on this change. As has been suggested herein there is historic evidence for this and is one reason why it is postulated that acid suppression has caused an increase in cancer. Again, as discussed above, provided this understanding of the role pH plays in these disease, a number of different novel strategies can be proposed to promote the cardiac to oxyntocardiac transformation and thereby inhibit the cardiac to intestinal conversion:

(a) New alternatives to the present acid suppressive drugs to treat reflux symptoms and complications;

(b) Drugs that inhibit or antagonize the alkalinizing effect of duodenogastric refluxate; and

(c) Cautious and limited use of acid suppressive drugs in the treatment of reflux disease.

In short, it is proposed that acid suppressive agents should be regarded as serious drugs with significant adverse effects including the promotion of intestinal metaplasia in cardiac mucosa, and they should be taken away from the realm of over-the-counter drugs that are marketed directly to the public by intense advertising and made prescription-only with adequate warnings regarding their use.

Finally, in another embodiment the current invention proposes to treat reflux disease by inhibiting the molecular mechanism that causes the transformation of cardiac to intestinal epithelium. Any drug that is developed that can inhibit or reverse this reaction or antagonize the agent that is involved will tend to decrease the incidence of adenocarcinoma. Developing such a drug will involve growing esophageal cardiac epithelial cells in tissue culture and exposing these to a variety of conditions and different molecules normally present in the gastric juice. The end-point of the experiment is the detection of goblet cell differentiation or CdX-1 and CdX-2 gene activation. Once these are identified, drugs can be developed that can either inhibit the agent that causes the transformation or effectively reverse the metaplastic molecular reaction. This will have the effect of converting intestinal epithelium to cardiac mucosa and preventing adenocarcinoma.

In summary, the failure to recognize these novel treatment avenues and continuing the single-minded development of acid suppressive agents guarantees that there will be no new drugs. Many of the suggested treatment avenues deal with simple molecular reactions and simple molecules that the pharmaceutical industry has considerable proven success in dealing with.

Surgical Methods of Preventing Adenocarcinoma

Many different surgical methods for preventing adenocarcinoma are progressing from the research arena into clinical application. The most advanced technique is anti-reflux surgery, usually a Nissen fundoplication where a valve effect is created at the end of the esophagus by wrapping the fundus of the stomach around the tubular esophagus. This procedure creates a new sphincter action that prevents gastroesophageal reflux when it is successful. The surgery is based on the principle that preventing reflux sequesters the esophageal epithelium from all molecules in gastric juice and prevents injury. A few controlled studies are emerging that successful anti-reflux surgery is effective in preventing the progression to adenocarcinoma. The most commonly performed anti-reflux procedure is surgical, usually laparoscopic. More recently, endoscopic techniques to create a barrier to reflux are also under study; these have limited proven success and acceptance at this time.

Methods of ablating Barrett epithelium are also gaining favor. When combined with effective control of reflux, the ablated surface can be induced to regenerate as squamous epithelium, decreasing cancer risk. Endoscopic mucosal resection and ablation with physical methods such as laser, electrocoagulation, and photodynamic therapy are increasing in effectiveness and acceptance. In general, these techniques are presently used mainly for Barrett esophagus complicated by high grade dysplasia or early adenocarcinoma in patients who are not surgical candidates. With increasing success, these techniques can be extended to uncomplicated Barrett esophagus.

The final target of surgical treatment is the control of duodenogastric reflux which is widely believed to be involved in carcinogenesis. Csendes et al have devised and reported a radical operation for the treatment of Barrett esophagus which includes separating the stomach from the duodenum and preventing gastroduodenal reflux by creating a Roux-en-Y loop. (Csendes, et al., 1993, previously cited.) This is still in an experimental phase and not performed with any frequency; however, provides a logical basis for preventing adenocarcinoma.

Although any of these techniques may be used, any surgical technique should be reserved as a last resort should no pharmaceutical approach prove effective.

Inhibiting Gastric Juice Events Responsible for Molecular Events

As discussed throughout, every molecular event that occurs in reflux disease, including reflux-induced adenocarcinoma, is induced by a molecule in gastric refluxate acting upon an esophageal epithelium. The gastric refluxate consists of gastric juice. Assuming that all the changes of reflux disease are caused by a luminally acting agent every injury-producing molecule must be present in the gastric juice. There is no evidence that any event in reflux disease is caused by a blood borne agent.

Research into identification of the molecules responsible for molecular events is complicated because there is the possibility that the agent is not constantly present. If the agent causes a molecular event and disappears, no amount of searching in gastric juice can identify the agent. As such, this type of research is often of limited value. However, within broad limits, the probability of identifying any causative agent is greatest when comparison is made between the gastric juice of those at highest and lowest risk for that event. Accordingly, in one embodiment of the invention, possible therapeutic targets are identified by careful examination of clinical groups can be used to identify the types of patient at highest and lowest risk for a given event.

(a) Squamous Epithelial Damage

There is excellent evidence that the early squamous epithelial changes are the result of acid exposure. Tobey et al. have shown that exposure of squamous epithelial cells in vitro to acid causes dilatation of intercellular spaces and increased permeability. (Tobey et al., 2004, previously cited.) Acute and severe injury that leads to erosion of the squamous epithelium (erosive esophagitis) is also certainly caused by acid, as evidenced by the rapid healing that is induced by acid suppressive drugs. It is not certain that other molecules in the refluxate are synergistic with acid in causing squamous epithelial damage. However, the practical importance of acid as the agent that causes squamous epithelial damage is strongly suggested by the historical decline in severe ulceration and strictures of squamous epithelium that has occurred with improving ability to pharmacologically suppress acid.

(b) Squamous to Cardiac Transformation

The specific interactive agent that transforms squamous epithelium to cardiac mucosa is unknown. This is the chronic manifestation of non-erosive reflux disease. The proven relationship between the amount of cardiac mucosa present and the severity of cumulative chronic reflux permits the practical recognition of patients at highest risk for cardiac metaplasia. The longer the cardiac mucosa and the younger the patient, the greater the likelihood that the environment favors the squamous to cardiac mucosal transformation.

There is also excellent correlation between the length of cardiac mucosa present in the esophagus (i.e. non-erosive reflux disease) and the 24-hr pH test. However, a correlation of a change with the 24-hr change does not prove that the change is caused by acid; even if the change is caused by another molecule that accompanies acid, the change will show a correlation with the 24-hr pH test. However, the fact that there is such a good correlation suggests that the agent that actually causes the squamous to cardiac transformation is ubiquitous in gastric juice. The transformation is the result of amount of reflux rather than any differences in the concentration of the agent.

Cardiac mucosa is a common finding in patients who have been on long-term acid suppressive drugs therapy. This does not prove anything because of the fact that the squamous to cardiac mucosal metaplasia occurs early in life; the start of acid suppressive therapy may have been after the transformation had already occurred. This finding only shows that acid suppression is very likely incapable of reversing the cardiac mucosa to squamous epithelium.

(c) The Cardiac to Oxyntocardiac Transformation

The interaction that causes the development of parietal cells in cardiac mucosa is likely the result of activation of a gastric-type genetic signal in the progenitor cell of cardiac mucosa. Like, the squamous to cardiac mucosal transformation, it is likely that the molecule responsible is ubiquitous in gastric juice. Oxyntocardiac mucosa appears to result from the existence of a milieu created by the amount and type of reflux rather than changes in the concentration of any molecule in gastric juice. Oxyntocardiac mucosa is frequently the only epithelial type seen in patients with reflux disease limited to end-stage dilated esophagus. In patients with visible columnar lined esophagus, oxyntocardiac mucosa is present largely in the more distal region. This suggests that the cardiac to oxyntocardiac transformation is the result of factors or molecules present at highest levels closest to the gastroesophageal junction. Possibilities include:
(1) Continuous exposure to gastric juice, as occurs in the dilated end-stage esophagus where oxyntocardiac mucosa dominates.
(2) Any molecule in gastric juice, because this will tend to have the highest concentration at the gastroesophageal junction.
(3) Low pH; high acidity tends to favor the cardiac to oxyntocardiac transformation. This is likely to be due to the fact that the interaction is pH dependent; searching for molecules in gastric juice that are more active at low pH is likely to detect the molecule responsible for this change.

(d) The Cardiac to Intestinal Metaplasia Transformation

In complete contrast to oxyntocardiac mucosa, intestinal metaplasia as well as goblet cell density is greatest in the most proximal part of the columnar lined segment immediately distal to the squamocolumnar junction. (Chandrasoma P T, Der R, Dalton P, Kobayashi G, Ma, Y, Peters J H, DeMeester T R. Distribution and significance of epithelial types in columnar lined esophagus. Am J Surg Pathol 2001; 25:1188-1193, the disclosure of which is incorporated herein by reference.) The fact that intestinal metaplasia has increased in prevalence over the past three decades suggests that this interaction is not caused by acid; the change is either not influenced by acid or potentiated by acid suppression. There is a suspicion that bile acid derivatives produced in the refluxate as a result of changes in pH caused by incomplete acid suppression may be responsible for this event. (Kazumori, 2005, previously cited.)

The fact that intestinal metaplasia increases in prevalence as the length of columnar lined esophagus increases, and becomes invariable at a given length suggests that the molecule responsible for this change is ubiquitous in gastric juice. In our study, 100% of patients with a columnar lined esophagus >5 cm had intestinal metaplasia. If the molecule causing the change was only present in some people, the prevalence of intestinal metaplasia would not reach 100% at any length of columnar lined esophagus. There is evidence in the literature that the length at which the prevalence of intestinal metaplasia reaches 100% has decreased in the past five decades. In Allison and Johnstone, many patients with columnar lined esophagus reaching the arch of the aorta did not have intestinal metaplasia. (Allison & Johnstone, 1953, previously cited.) By 1976 (Paull et al., previously cited), it seems that intestinal metaplasia was seen in the majority of patients with a 10 cm length of columnar lined esophagus. Herein it is proposed that this increased tendency to develop intestinal metaplasia at increasingly lower levels is the result of alkalinization of gastric juice.

The fact that intestinal metaplasia has a maximal likelihood of occurring at a point furthest away from the gastroesophageal junction suggests that the factors favoring this transformation are dependent on the molecule interacting with the progenitor cell at a given pH. This could be the result of two independent things:
(1) The concentration of the factor in gastric juice is increased so that it reaches a higher point in the esophagus during reflux, and\
(2) The pH of the gastric juice is increased so that the correct pH is present at a lower level in the esophagus during reflux. There is also the possibility that the transformation is favored by the higher damage level associated with the intermittent, pulse-type exposure of the epithelium to the refluxate that is likely in the higher regions of the esophagus.

If patients are classified into those with low and high tendency to cause the cardiac to intestinal mucosal transformation, the outcomes of these patients in terms of the likelihood of Barrett esophagus at different lengths of cardiac mucosa in the esophagus can be summarized, as shown in Table 10, below.

TABLE 10
EFFECT OF DAMAGE ON TENDENCY FOR INTESTINAL
METAPLASIA (IM).

| Damage | | |
|---|---|---|
| Level | IM tendency | Clinical Effect |
| Low | Low | Microscopic CLE with CM and OCM |
| Intermediate | Low | <2 cm OLE with CM and OCM |
| High | Low | 2-5 cm CLE with CM and OCM |
| Very high | Low | >5 cm CLE with IM restricted to |
| | | proximal end of CLE |
| Low | High | Microscopic CLE with IM |
| Intermediate | High | Short segment CLE with IM (SSBE) |
| High | High | Long segment CLE with IM (LSBE) |
| Very high | High | >5 cm CLE with IM involving most of |
| | | CLE |

By this reasoning, patients with intestinal metaplasia in the shortest segments of columnar lined esophagus are those in whom the factors favoring the cardiac to intestinal transformation are greatest (FIG. 29). Comparing the group at highest risk for intestinal metaplasia with the group that has the least risk (i.e. patients with the longest segments of columnar lined mucosa that do not have intestinal metaplasia) is likely to detect which factors and molecules in the gastric refluxate are responsible for intestinal metaplasia.

This is counter-intuitive; while damage by reflux produces increasing lengths of columnar lined esophagus, the factors causing intestinal metaplasia are maximal in those patients who have the shortest segments of Barrett esophagus. In these patients, the intestinal metaplasia has occurred in cardiac mucosa present at a more distal point in the esophagus at a more acidic pH and with a lesser pulse effect than patients with long segment Barrett esophagus (FIG. 29). This new mechanistic understanding produces an expectation that is very different from the conventional view.

This theory can explain the differences in gender distribution that is seen with different lengths of Barrett esophagus. If females have a greater tendency to convert the cardiac mucosa to intestinal metaplasia than males, females will develop intestinal metaplasia with shorter segments of cardiac metaplasia than males. This assumes that once intestinal metaplasia has occurred in a segment of columnar lined esophagus, it ceases to increase in length, which I have suggested is the case.

The critical factor to assess when trying to evaluate the tendency to intestinal metaplasia is not the overall prevalence of intestinal metaplasia or the overall length of Barrett esophagus in males and females. Both of these are much greater in males. The critical measure is the ratio between patients with long and short Barrett esophagus. Short segment Barrett esophagus is much more frequently seen than long segment Barrett esophagus in women; long segment Barrett esophagus is much more common in men. By having a SSBE:LSBE ratio that is much higher, women would be identified as the gender with the greater tendency to develop intestinal metaplasia.

Studying the differences in gastric contents between women who have microscopic segments of Barrett esophagus (i.e. presently diagnosed as intestinal metaplasia of the gastric cardia) and males with long segments of columnar lined esophagus who do not have intestinal metaplasia is valuable because these two groups represent the two extremes in terms of tendency to induce intestinal metaplasia FIG. 21; Table 3). Accordingly, in one embodiment of the invention treatment methodology is determined through the identification of molecules in the gastric juice that show the greatest difference in concentration in these two groups because it is likely to lead to the agent causing intestinal metaplasia in cardiac mucosa.

(e) Carcinogenesis in Intestinal Metaplasia

Many factors are involved in carcinogenesis:

(1) The presence of carcinogen(s). The very low incidence of adenocarcinoma in reflux disease suggests that the carcinogen in not ubiquitous in gastric juice. The carcinogen(s) may be endogenous secretions of the foregut or exogenous molecules in food. If the patient has duodenograstric reflux, the endogenous compounds available greatly increase. All these molecules interact in the stomach to produce new molecules that may be transient, disappearing after they have induced carcinogenesis. Whatever the carcinogenic molecule(s), it is self evident that they are present at highest concentration in the stomach, reach the esophagus by reflux, and decrease in concentration from the gastroesophageal junction to the more proximal esophagus (FIG. 30). The effective carcinogen concentration is always greatest in the most distal esophagus and reaches a variable height in the esophagus (A-D in FIG. 30).

(2) The presence or absence of the target cell. The target cell for reflux-induced adenocarcinoma is the actively proliferating cell in intestinal metaplastic epithelium. If there is no intestinal metaplasia, there is no risk of malignancy irrespective of the length of columnar lined esophagus. Of course, there is the possibility that the target cell may appear at a point in the future in a given patients. The absence of the target cell in a 50 year old patient is much more highly predictive of a lack of cancer risk than the absence of intestinal metaplasia in a child. The presence of the target cell indicates risk of cancer, irrespective of length of columnar lined esophagus.

(3) The number of target cells present: This is an unevaluated risk, but it is logical to believe that the cancer risk will be directly proportional to the number of target cells present because the likelihood of a mutational event will be proportional to the number of targets. As discussed herein, re-classification of Barrett esophagus by some measure of the number of target cells will provide better risk delineation that the present classification into long and short segment Barrett esophagus.

(4) The location of the target cells: Because the concentration of the carcinogen is highest distally and the occurrence of intestinal metaplasia is maximal in the most proximal region of the segment of columnar lined esophagus, it is essential that the intestinal metaplasia reach low enough to encounter sufficient carcinogen (FIGS. 19 and 20).

(5) The proliferative rate of the target cell: Intestinal epithelium has the highest proliferative rate among the different epithelial types in columnar lined esophagus. (Olvera et al, 2005, the disclosure of which is incorporated herein by reference.) It is logical that cancer risk will correlate with proliferative rate because the probability of mutations increases with increasing proliferative rate.

3. Although the exact molecular mechanism(s) of carcinogenesis is unknown, it is proposed that bile salt metabolites maybe involved (see Chapter 10). The observed rates of progression from reflux to dysplasia and cancer are variable in patients with Barrett esophagus. Currently these patients are placed on acid suppression (if symptomatic) and regular surveillance at the time of diagnosis. At the time they are placed on surveillance, the duration for which intestinal metaplasia has been present is unknown. Some of these patients develop cancer within 1 year of the index endoscopy. This is a high carcinogenic group. The risk of cancer in the remainder of the patients is 0.5 percent per year and is greatest in patients who have evidence of progression in the carcinogenic pathway at the index biopsy, e.g., have low grade dysplasia (Weston et al) or aneuploidy (Reid et al). (Weston A P, Badr A S, Hassanein R S. Prospective multivariate analysis of clinical, endoscopic, and histological factors predictive of the development of Barrett's multifocal high grade dysplasia or adenocarcinoma. Am J Gastroenterol 1999; 94:3413-3419; and Reid B J, Levine D S, Longton G, et al. Predictors of progression to cancer in Barrett's esophagus: Baseline histology and flow cytometry identify low- and high-risk patient subset. Am J Gastroenterol 2000; 95:1669-1676, the disclosures of which are incorporated herein by reference.) The majority of patients who are on surveillance for Barrett esophagus have very stable disease without progression to increasing dysplasia or cancer over many years. This clinical course identifies a low carcinogenic environment; there is no progression to cancer despite the presence of large numbers of target cells.

Most patients who develop reflux induced adenocarcinoma have been completely asymptomatic or take over-the-counter acid suppressive agents for mild symptoms. They present for the first time with symptoms related to the cancer. The rate of progression in this group is unknown because it is not known when they developed intestinal metaplasia in the absence of surveillance.

Based on these observations, it is possible to predict the likelihood of carcinogenesis based on differences in the elements of damage (which controls length of columnar lined esophagus), tendency to intestinal metaplasia (which controls the number and location of target cells), and carcinogenicity that we have discussed above. These elements are summarized in Table 11, below.

TABLE 11
PATTERNS OF ADENOCARCINOMA

| | Intestinal | | |
|---|---|---|---|
| Damage | metaplasia | Carcinogenic | |
| environment | tendency | environment | Characteristics in patient |
| Low | Low | Low | Short CLE; asymptomatic; |
| | | | Late age onset of IM; |
| | | | No/slow progression to cancer |
| Low | Low | High | Short CLE; asymptomatic; |
| | | | Late age onset of IM; |
| | | | Rapid progression to cancer |
| Low | High | Low | Short CLE; asymptomatic |
| | | | Early age onset of IM; |
| | | | No/slow progression to cancer |
| Low | High | High | Short CLE; asymptomatic; |
| | | | Early age onset of IM; |
| | | | Rapid progression to cancer |
| High | Low | Low | Long CLE; symptomatic; |
| | | | Late age onset of IM; |
| | | | No/slow progression to cancer |
| High | Low | High | Long CLE; symptomatic; |
| | | | Late age onset of IM; |
| | | | Rapid progression to cancer |
| High | High | Low | Long CLE; symptomatic |
| | | | Early age onset of IM; |
| | | | No/slow progression to cancer |
| High | High | High | Long CLE; symptomatic |
| | | | Early age onset of IM; |
| | | | Rapid progression to cancer |

This shows that the length of columnar lined esophagus and the presence of symptoms is a function of the damage environment; the age of onset of intestinal metaplasia is a function of the tendency to produce intestinal metaplasia; and the progression to cancer from the time of onset of intestinal metaplasia is a function of the carcinogenic environment. These are all likely to be independent mechanisms with at least the tendency to intestinal metaplasia and carcinogenicity being unrelated to acid.

This construct fits well with observed patterns of reflux-induced adenocarcinoma if the dominant factor for the development of cancer is the carcinogenicity of the refluxate. In a given individual with a highly carcinogenic refluxate, the following things may happen:
(a) If the patient has a low damage environment and a low tendency to develop intestinal metaplasia, that patient may never develop the critical length of columnar lined esophagus to develop intestinal metaplasia. Without intestinal metaplasia, the patient will not develop cancer. This individual will not have symptoms of reflux and at the time of natural death will have a very short segment of cardiac and/or oxyntocardiac mucosa.
(b) If the patient has a low damage environment with a strong tendency to develop intestinal metaplasia, the patient will not have symptoms but will develop very short segment Barrett esophagus at a relatively young age. Because the carcinogenicity is high, this patient represents the very common case of an individual who develops adenocarcinoma in the end-stage dilated esophagus without developing symptoms and without detection of Barrett esophagus even if an endoscopy is done.

In contrast, the patient with a low carcinogenic environment will progress to cancer very slowly or not at all. These patients will have a length of columnar lined esophagus and symptoms based on their damage environment. If they develop intestinal metaplasia and are so diagnosed, they will be the population of patients with Barrett esophagus who remain stable on surveillance over long periods without progressing to cancer.

Recognizing the factors that induce carcinoma in intestinal metaplasia are best determined by evaluating differences in the composition of gastric juice in those at lowest and highest risk for cancer. Unlike the tendency to intestinal metaplasia, which is highest in patients who develop intestinal metaplasia in the shortest segments of cardiac mucosa, the tendency to carcinogenesis in intestinal metaplasia are greatest in patients who develop cancer in the most proximal esophagus at the youngest age (FIG. 30). The patient who has a high damage element and minimal tendency to intestinal metaplasia will develop intestinal metaplasia restricted to the most proximal region of a long segment of columnar lined esophagus (patient #1 in FIG. 30). This patient can develop cancer only in the most proximal segment of the columnar lined esophagus where target cells are present, and only if there is a high concentration of carcinogen (A in FIG. 30). The patient with the highest concentration of carcinogenic molecules in the gastric juice is therefore identified as the youngest patient who develops the most proximal adenocarcinoma in a very long segment of columnar lined esophagus with intestinal metaplasia limited to its most proximal region (patient #1 in FIG. 30).

In contrast, the lowest concentration of carcinogen is most likely in the oldest patient who has a long segment of columnar lined esophagus with intestinal metaplasia reaching the most distal esophagus who has remained stable for long periods without developing dysplasia or cancer during surveillance. This patient develops intestinal metaplasia in the distal esophagus and has the highest number of target cells for the carcinogen to act at a point that is close to the highest carcinogen concentration (patient #2 in FIG. 30). The fact that this patient does not develop dysplasia and cancer over a long period can only be explained by low carcinogenic environment.

It should be realized that these two patients with the highest and lowest carcinogenicity will both be classified by present criteria as long segment Barrett esophagus. It is only when it is recognized that patients with long segment Barrett esophagus have infinitely variable amounts of intestinal metaplasia with consequent differences in the risk of cancer that any sense can be made of this disease.

Treatment methodologies that focus on molecules present at higher concentration in the gastric juice of patients with a high compared to low carcinogenic tendency will be more likely to be the true carcinogen(s). Once these are characterized, their ability to induce carcinogenesis can be tested in experimental cell culture systems. This method is likely to be far more effective in characterizing the actual carcinogenic molecule(s) than the present shotgun approach to studying gastric juice molecules.

Molecular Research into Carcinogenesis

There is extensive research being conducted at present into attempting to determine the molecular pathway of carcinogenesis in Barrett esophagus. Many molecular abnormalities have been identified in Barrett esophagus as well as dysplasia and adenocarcinoma. Putting these molecular changes together to work out the exact molecular/genetic pathway of carcinogenesis in Barrett esophagus is on the near horizon.

Unfortunately, it is not likely that understanding the molecular pathway of Barrett esophagus carcinogenesis will have practical value any time soon. We have understood the molecular/genetic pathway in colorectal cancer for more than a decade now but this has not impacted significantly in the management and prevention of colorectal cancer. Reversal of molecular events responsible for carcinogenesis awaits the development of genetic manipulation and delivery of gene therapy agents. As more time passes without success in these areas, the probability that they will not succeed increases.

In contrast, there is very little research directed at the molecular events in the pre-Barrett metaplastic stage of the disease. This is because of the false dogma that exists that cardiac mucosa is a normal gastric epithelium. Recognition that cardiac mucosa is always abnormal, as proposed in the current invention allows for a molecular intervention of this epithelial type. An appropriate treatment technique could arise from any of the following:

(a) First addressing the genotype of cardiac epithelial cells and how this differs from squamous epithelium. This provides insight into the mechanism by which squamous epithelium transforms into cardiac mucosa and the genetic signal that causes the progenitor cell to differentiate into squamous or cardiac epithelium, thereby providing one possible treatment strategy.

(b) Examining what differences exist in the genetic make up of cardiac and intestinal epithelium. There is evidence that Cdx genes are important in the transformation of cardiac to intestinal epithelium. In one treatment regime agents can be directed at the interaction between the target cell in cardiac epithelium and the molecule in gastric refluxate, the receptor on the cell, the molecule in the refluxate, and the steps between the receptor binding with the molecule and the genetic change.

(c) Finally, the differences that exist in the genetic make up of cardiac and oxyntocardiac epithelium. There is evidence that the sonic Hedgehog gene may be important in the transformation of cardiac to oxyntocardiac epithelium. A similar series agents to those discussed above could be directed to interfering with this transformation process.

Summary

Regardless of the treatment regime chosen, the first crucial step identified by the current invention is the understanding of the cellular processes involved. The failure to do this in reflux disease has resulted in much misdirected research. By identifying the molecular mechanism that converts cardiac mucosa to oxyntocardiac mucosa it becomes possible to induce this change through treatment regimes. In such a treatment all that is required to prevent intestinal metaplasia is to screen people for cardiac mucosa in accordance with the current invention, and remove them from the reflux-adenocarcinoma sequence by inducing oxyntocardiac mucosa. Alternatively, a drug could be administered to inhibit or reverse the molecular step involved in the conversion of cardiac mucosa to intestinal metaplasia, which would have a similar effect in preventing esophageal adenocarcinoma.

Such a molecular strategy would appear much more feasible than gene therapy directed at esophageal dysplasia or cancer. Moreover, there is precedent for such success: identification of the molecular mechanism associated with CD117 and tyrosine kinase receptors permitted the development of Gleevac, a drug directed at this molecular interaction that effectively controls the growth of CD117+ gastrointestinal stromal tumors. The objective in the current invention is to identify potential drugs to intervene in the early metaplastic stage of reflux induced esophageal carcinogenesis. For the purposes of this invention, it should be understood that reflux-induced adenocarcinoma includes adenocarcinoma arising in the dilated end-stage esophagus, which is presently mis-classified as adenocarcinoma of the gastric cardia.

### CONCLUSION

The novel gastroesophageal reflux disease diagnostic and treatment methodologies of the current invention are superior to any conventional measure. Moreover, because the main complication of reflux disease is adenocarcinoma, the current invention also relates to the diagnosis and treatment of this advanced form of the disease.

While preferred embodiments of the foregoing invention have been set forth for purposes of illustration, the foregoing description should not be deemed a limitation of the invention herein. Accordingly, various modifications, adaptations and alternatives may occur to one skilled in the art without departing from the spirit and scope of the present invention.

What is claimed is:

1. A method of diagnosing a gastroesophageal disorder comprising:
examining at least one of either the esophageal epithelial or the gastric mucosa of a patient;
determining the presence or absence of the gastroesophageal disorder based on a histological observation of the condition of the at least one of either the esophageal epithelial or the gastric mucosa of the patient.

2. The method of claim 1, wherein the disorder is reflux carditis, and wherein a diagnosis of reflux carditis is made where at least one of the following conditions are found: the presence of a segment of abnormal epithelium along at least one portion of the esophagus or the presence of a segment of abnormal mucosa along at least one distal portion of the esophagus proximal to the stomach.

3. The method of claim 2, wherein the abnormal epithelium is any epithelium type other than a squamous epithelium.

4. The method of claim 3, wherein the abnormal epithelium is a metaplastic columnar epithelium.

5. The method of claim 2, wherein the abnormal epithelium is selected from one of either cardiac mucosa or oxynto-cardiac mucosa.

6. The method of claim 4, wherein the segment of metaplastic columnar epithelium is greater than about 2 cm.

7. The method of claim 5, wherein the segment of cardiac mucosa or oxynto-cardiac mucosa is greater than about 1 mm.

8. The method of claim 5, wherein the cardiac mucosa contains a plurality of mucosa cells showing a chronic inflammation and a reactive change.

9. The method of claim 8, wherein the chronic inflammation includes frequent eosinophils, plasma cells and lymphocytes.

10. The method of claim 8, wherein the reactive change includes one of the following: gland distortion, foveolar hyperplasia, and fibrosis and smooth muscle proliferation in the lamina propria.

11. The method of claim 8, wherein the cardiac mucosa produces polypoid mucosal lesions at the gastroesophageal junction.

12. The method of claim 1, further including conducting a 24 hour pH test.

13. The method of claim 1, further including examining the patient endoscopically for a flat columnar mucosa between a proximal limit of the rugal folds and squamous epithelium.

14. The method of claim 1, further including taking a biopsy of the region immediately distal the squamous epithelium.

15. The method of claim 14, wherein the biopsy step follows a Demeester biopsy protocol.

16. The method of claim 1, further including monitoring the patient for Helicobacter pylori gastritis by examining the gastric mucosa for inflammation.

17. The method of claim 2, further comprising determining the severity of the reflux disease by measuring the length of the segment of abnormal epithelium.

18. The method of claim 17, wherein mild reflux disease is characterized by a microscopic segment of a metaplastic columnar epithelium between the squamous epithelium and the gastric oxyntic mucosa.

19. The method of claim 17, wherein moderate reflux disease is characterized by a <2 cm segment of a metaplastic columnar epithelium between the squamous epithelium and the gastric oxyntic mucosa.

20. The method of claim 17, wherein severe reflux disease is characterized by a >2 cm segment of a metaplastic columnar epithelium between the squamous epithelium and the gastric oxyntic mucosa.

21. The method of claim 1, wherein the disorder is Barrett's esophagus, and wherein a diagnosis of Barrett esophagus is made where the following conditions are found: a segment of cardiac mucosa is present along at least one distal portion of the esophagus proximal to the stomach, and one or more goblet cells are present in the cardiac mucosa.

22. The method of claim 21, wherein the examination includes taking a stain of a section of the esophagus.

23. The method of claim 22, wherein the stain is a hematoxylin and eosin stain.

24. A method of diagnosing a patient's risk of developing reflux-induced adenocarcinoma of the esophagus comprising:
determining the presence or absence of a columnar lined esophagus and the length thereof;
identifying the nature of the abnormal epithelia in the columnar lined esophagus;
measuring the pH gradient from the gastroesophageal junction to the proximal esophagus;
determining the presence or absence of intestinal metaplasia and the location thereof along the esophagus; and
determining whether the patient has gastroesophageal reflux disease.

25. The method of claim 24, wherein the patient is determined to be at higher risk of cancerous growth where a columnar lined esophagus is present having a epithelium comprising cardiac mucosa.

26. The method of claim 24, wherein the patient is determined to be at higher risk of cancerous growth with the increasing length of the cardiac mucosa.

27. The method of claim 26, wherein the length of the cardiac mucosa is >2 cm.

28. The method of claim 24, wherein the patient is determined to be at higher risk of cancerous growth where the pH of the esophagus is alkaline.

29. The method of claim 28, wherein the pH of the patient's gastric juice is in a range of about 3 to 6.

30. The method of claim 24, wherein the patient is determined to be at higher risk of cancerous growth where intestinal metaplasia is present

31. The method of claim 24, wherein the patient is determined to be at higher risk of cancerous growth where the intestinal metaplasia is located in the distal portion of the esophagus.

32. The method of claim 24, wherein the patient is determined to be at higher risk of cancerous growth where the patient has gastroesophageal reflux disease.

33. The method of claim 24, wherein the patient is determined to be at low risk of cancerous growth where the epithelium of the columnar lined esophagus is oxynto-cardiac mucosa.

34. The method of claim 24, wherein the structural determinations include taking at least one biopsy of the esophagus.

35. The method of claim 34, wherein the biopsy is taken in the region of the esophagus immediately distal to the squamo-columnar junction.

36. The method of claim 34, wherein a plurality of biopsies are taken along the length of the columnar-lined esophagus.

37. The method of claim 24, further comprising monitoring the concentration of highly ionic refluxates in the esophagus of the patient.

38. The method of claim 37, wherein the highly ionic refluxates are selected from the group consisting of: acid, bile salt, and a derivative of bile acid metabolism.

39. The method of claim 24, wherein the carcinogenesis of interest is one of either dysplasia or adenocarcinoma.

40. A method of treating a gastroesophageal disorder in a patient comprising:
providing a therapeutic course of treatment to prevent or reverse a disorder triggered transformation of a healthy esophageal epithelium to an unhealthy esophageal epithelium.

41. The method of claim 40, wherein the healthy epithelium is a squamous epithelium.

42. The method of claim 40, wherein the treatment comprises a genetic therapy to reverse the genetic switch that triggers the transformation of the healthy esophageal epithelium to the unhealthy esophageal epithelium.

43. The method of claim 42, wherein the transformation is from a squamous epithelium to an intestinal metaplasia through an intermediate epithelium.

44. The method of claim 43, wherein the intermediate epithelium is cardiac mucosa

45. The method of claim 42, wherein the disorder triggered transformation is the development of at least one goblet cell, and wherein the genetic switch involves the CdX gene system.

46. The method of claim 40, wherein the treatment includes a therapy to lower the pH of the patient's gastric juice.

47. The method of claim 46, wherein the pH is maintained in a range of between about 1 to 3.

48. The method of claim 40, wherein the treatment comprises a therapy to promote the production of a concentration of parietal cells in a metaplastic columnar epithelium such that a cardiac mucosa epithelium is converted to an oxynto-cardiac mucosa epithelium.

49. The method of claim 48, wherein the therapy includes a genetic therapy to promote the genetic switch which induces the production of parietal cells in the cardiac mucosa.

50. The method of claim 49, wherein the genetic switch involves the Sonic Hedgehog gene.

51. The method of claim 40, wherein the treatment comprises suppressing at least one class of molecule that promotes the transformation of a squamous epithelium to a cardiac mucosa or an intestinal metaplasia.

52. The method of claim 51, wherein the at least one class of molecule is a bile salt derivative selected from the group consisting of: cholic acid and dehydrocholic acid.

53. The method of claim 40, wherein the treatment includes suppressing esophageal pain by promoting the transformation of a reflux-damaged squamous epithelium to a columnar epithelia.

54. The method of claim 53, wherein the columnar epithelia is an oxynto-cardiac mucosa.

55. The method of claim 40, wherein the treatment includes promoting the transformation of a cardiac mucosa to an oxynto-cardiac mucosa.

56. The method of claim 55, wherein the treatment includes administering an agent to lower the pH of the esophageal environment without a concomitant increase in the gastric pH.

57. The method of claim 56, wherein the treatment includes administering an agent to suppress duodenogastric reflux and antagonize the alkalinity of any duodenogastric reflux.

58. The method of claim 40, wherein the treatment is designed to prevent a transformation by intervening in at least one of the mechanisms selected from the group consisting of: inhibiting cardiac mucosal metaplasia of squamous epithelia, inducing squamous differentiation, promoting cardiac metaplasia in damaged squamous epithelium, promoting conversion of cardiac mucosa to oxynto-cardiac mucosa, inhibiting conversion of cardiac mucosa to intestinal metaplasia, and reversing intestinal metaplasia to cardiac mucosa.

59. A method of screening therapeutic compounds effective for treating a gastroesophageal disorder, comprising:
exposing at least one sample of epithelial to a plurality of molecular components in gastric juice;
monitoring said at least one sample for columnar cellular transformation.

60. The method of claim 59, wherein the cellular transformation monitored is the transformation from cardiac mucosa to oxynto-cardiac mucosa.

61. The method of claim 59, further comprising comparing the activated genes in the transformed sample to determine the identity of the gene activated in the metaplastic switch.

62. The method of claim 61, wherein at least one of the genes to be monitored is the Sonic Hedgehog gene.

63. The method of claim 59, further comprising screening at least two epithelial samples taken from patients at high and low risk for carcinogenesis and comparing the results.
